(19) United States
(12) Patent Application Publication
(10) Pub. No.: US 2017/0333404 A1
(43) Pub. Date: Nov. 23, 2017

(54) IMPROVED FORMULATIONS OF VEMURAFENIB AND METHODS OF MAKING THE SAME

(71) Applicant: DISPERSOL TECHNOLOGIES, LLC, Georgetown, TX (US)

(72) Inventors: Dave Alan MILLER, Round Rock, TX (US); Justin Martin KEEN, Round Rock, TX (US); Chris BROUGH, Austin, TX (US); Sandra U. KUCERA, Cedar Park, TX (US); Daniel J. ELLENBERGER, Austin, TX (US)

(73) Assignee: DISPERSOL TECHNOLOGIES, LLC, Georgetown, TX (US)

(21) Appl. No.: 15/523,832

(22) PCT Filed: Nov. 3, 2015
(86) PCT No.: PCT/US15/58742

§ 371 Date: May 2, 2017

Related U.S. Application Data
(60) Provisional application No. 62/074,465, filed on Nov. 3, 2014.

(51) Int. Cl. A61K 31/437; A61K 9/16; A61K 9/14; B01F 7/00; A61K 31/18

(52) U.S. Cl. CPC A61K 31/437; B01F 7/001; A61K 31/18; A61K 9/146; A61K 9/1605; A61K 9/1694

(57) ABSTRACT

The disclosure provides for improved pharmaceutical compositions containing vemurafenib and methods of manufacturing the same. In particular, the compositions are prepared using thermokinetic compounding and provide improved properties as well as more efficient methods of manufacture.

This application claims benefit of priority to U.S. Provisional Application Ser. No. 62/074,465, filed Nov. 3, 2014, the entire contents of which are hereby incorporated by reference.

### BACKGROUND OF THE INVENTION

### 1. Field of the Invention

The present disclosure relates in general to the field of pharmaceutical preparation and manufacturing, and more particularly, pharmaceutical formulations of vemurafenib using thermokinetic compounding.

### 2. Description of Related Art

The beneficial applications of many potentially therapeutic molecules is often not fully realized either because they are abandoned during development due to poor pharmacokinetic profiles, or because of suboptimal product performance. Alternatively, even if produced, the cost associated with formulating such molecules may create barriers to their widespread use. Problems with formulation are often due to poor solubility, resulting in poor bioavailability, increased expense, and ultimately termination of the product's development. In recent years, the pharmaceutical industry has begun to rely more heavily on formulational methods for improving drug solubility. Consequently, advanced formulation technologies aimed at enhancing the dissolution properties of poorly water soluble drugs are becoming increasingly important to modern drug delivery.

Vemurafenib is a BRAF kinase inhibitor used to treat patients with metastatic melanoma with the BRAF V600E mutation. Because vemurafenib has a very high melting point, melt extrusion is not a practical commercial process for manufacturing. It is also poorly soluble in volatile organic solvents, eliminating spray drying as a process of manufacture. As such, at present it can only be produced using a costly and inefficient microprecipitated bulk powder (“MBP”) method. The resulting product costs about $25,000 per year.

Thus, while vemurafenib has significant therapeutic value for melanoma patients, it also exhibits extremely challenging properties with respect to pharmaceutical formulation. As a result, there is a great need in to provide improved compositions and methods of manufacturing for this drug.

### SUMMARY OF THE INVENTION

Thus, in accordance with the present disclosure, there is provided a method of making a pharmaceutical composition comprising (a) providing vemurafenib, or a pharmaceutically acceptable salt, ester, derivative, analog, prodrug or solvate thereof, and one or more pharmaceutically acceptable excipients; (b) compounding the materials of step (a) in a thermokinetic mixer for less than 300 seconds, wherein the thermokinetic compounding of vemurafenib and the one or more pharmaceutically acceptable excipients forms a melt blended pharmaceutical composite. The pharmaceutical may comprises a one or more different active pharmaceutical ingredients in addition to vemurafenib. The one or more pharmaceutically acceptable excipient may comprise a surfactant and/or a pharmaceutical polymer, including one or more surfactants and one or more polymer carriers.

The pharmaceutically acceptable excipient may comprise an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate.

The pharmaceutical polymer may comprise an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

The surfactant may comprise an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, and sorbitan laurate, and the pharmaceutical polymer comprises an agent selected from a group consisting of poly(vinylpyrrolidone), ethylacrylate-methylmethacrylate copolymer, poly(methacrylate ethylacrylate) (1:1) copolymer, hydroxypropylmethylcellulose acetate succinate, poly(butyl methacylate-co-(2-dimethylaminoethyl) methacrylate-co-methyl methacrylate) 1:2:1 and polyvinyl caprolactam-polyvinyl acetate-polyethylene glycol graft copolymer.

The method may further comprise providing and compounding a MEK inhibitor with vemurafenib. The MEK inhibitor may be is GDC-0973 or cobimetinib.

The one or more pharmaceutically acceptable excipients may comprise a processing agent, such as a plasticizer.

Step (b) may be performed at a maximum temperature of about 250° C., about 225° C., about 200° C., about 180° C., about 150° C., about 150° C. to 250° C., or about 180° C. to 250° C.

The one or more pharmaceutically acceptable excipients may comprise a non-ionic pharmaceutical polymer, such as a water soluble, cellulosic, or cellulosic and water soluble polymer. The non-ionic, water soluble pharmaceutical polymer may be poly(vinyl acetate)-co-poly(vinylpyrrolidone) copolymer, poly(vinlypyrrolidone), hydroxypropylcellulose, poly(vinyl alcohol), hydroxypropyl methylcellulose, hydroxyethylcellulose, polyvinyl caprolactam-polyvinyl acetate-polyethylene glycol graft copolymer and sodium carboxymethyl-cellulose.

The one or more pharmaceutically acceptable excipients may comprise a cross-linked pharmaceutical polymer. The cross-linked pharmaceutical polymer may be carbomer, crospovidone, or croscarmellose sodium.

The one or more pharmaceutically acceptable excipients may comprise a pharmaceutical polymer of high melt viscosity and/or a thermally labile pharmaceutical polymer. The one or more pharmaceutically acceptable excipients may comprise hypromellose acetate succinate.

The vemurafenib to pharmaceutical polymer ratio may be about 1 to 4, may be about 3 to 7, may be about 2 to 3, may be about 1 to 1.

In another embodiment, there is provided a pharmaceutical composition comprising an amorphous dispersion of vemurafenib, or a pharmaceutically acceptable salt, ester, derivative, analog, prodrug or solvate thereof, and one or more pharmaceutically acceptable excipients, wherein the one or more pharmaceutically acceptable excipients comprises a non-ionic pharmaceutical polymer. The pharmaceutical may comprise one or more active pharmaceutical ingredients in addition to vemurafenib, such as a MEK inhibitor, such as cobimetinib or GDC-0973.

The one or more pharmaceutically acceptable excipient may comprise a surfactant, a processing agent, or a plasticizer.

The pharmaceutically acceptable excipient may comprise an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate.

The pharmaceutical polymer may comprise an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

The surfactant may comprise an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, and sorbitan laurate, and the pharmaceutical polymer comprises an agent selected from a group consisting of poly(vinylpyrrolidone), hydroxypropylcellulose, poly(vinyl alcohol), hydroxypropyl methylcellulose, hydroxyethylcellulose, and sodium carboxymethyl-cellulose and polyvinyl caprolactam-polyvinyl acetate-polyethylene glycol graft copolymer.

The pharmaceutically acceptable excipient may comprise an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, sorbitan laurate, poly(vinyl acetate)-co-poly(vinylpyrrolidone) copolymer, hydroxypropylcellulose, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, and polyvinyl caprolactam-polyvinyl acetate-polyethylene glycol graft copolymer.

The pharmaceutical composition may not contain a processing agent, and/or may not contain a plasticizer. The composition may be a composite and is a homogenous, heterogeneous, or heterogeneously homogenous composition. The non-ionic pharmaceutical polymer may be a water soluble polymer, such as a water soluble polymer is selected from the group consisting of poly(vinlypyrrolidone), poly(vinyl acetate)-co-poly(vinylpyrrolidone) copolymer, hydroxypropylcellulose, poly(vinyl alcohol), hydroxypropyl methylcellulose, hydroxyethylcellulose, polyvinyl caprolactam-polyvinyl acetate-polyethylene glycol graft copolymer and sodium carboxymethyl-cellulose.

The pharmaceutical composition may have a vemurafenib to non-ionic pharmaceutical polymer ratio of about 1 to 4, about 3 to 7, about 2 to 3, or about 1 to 1.

The one or more pharmaceutically acceptable excipients may comprise a pharmaceutical polymer of high melt viscosity, and or a thermally labile pharmaceutical polymer.

The peak solubility of the vemurafenib in the composition may be greater than 10 μg/mL in an aqueous buffer with a pH range of 4 to 8. The peak solubility of vemurafenib and the reference standard vemurafenib in an aqueous buffer with a pH range of 4 to 8 have a ratio of greater than 3:1. greater than 10:1, greater than 20:1 or greater than 30:1. The Cₘₐₓ of the vemurafenib in the composition and Cₘₐₓ of the reference standard vemurafenib have a ratio that is greater than 6:1.

The pharmaceutical composition may be formulated into an oral dosage form, such as a tablet, a capsule, or a sachet.

Also provided is a pharmaceutical composition comprising an amorphous dispersion of vemurafenib and one or more pharmaceutically acceptable excipients, wherein the one or more pharmaceutically acceptable excipients comprises cross-linked pharmaceutical polymer. The cross-linked pharmaceutical polymer may be carbomer, crospovidone, polycarbophil, or croscarmellose sodium.

In yet a further embodiment, there is provided a pharmaceutical composition produced by a process comprising the steps of (a) providing vemurafenib and one or more pharmaceutically acceptable excipients; (b) compounding the materials of step (a) in a thermokinetic mixer for less than 300 seconds and at less than about 250° C., wherein the thermokinetic compounding of vemurafenib and the one or more pharmaceutically acceptable excipients forms a melt blended pharmaceutical composition. The one or more pharmaceutically acceptable excipients may include a non-ionic pharmaceutical polymer, a water soluble pharmaceutical polymer, cellulosic pharmaceutical polymer, a non-ionic, water soluble pharmaceutical polymer, a non-ionic, cellulosic pharmaceutical polymer, a water soluble, cellulosic pharmaceutical polymer, a thermally labile pharmaceutical polymer, a high melt viscosity pharmaceutical polymer, and/or a cross-linked pharmaceutical polymer.

The non-ionic, water soluble pharmaceutical polymer may be poly(vinlypyrrolidone), poly(vinyl acetate)-co-poly(vinylpyrrolidone) copolymer, hydroxypropylcellulose, poly(vinyl alcohol), hydroxypropyl methylcellulose, hydroxyethylcellulose, polyvinyl caprolactam-polyvinyl acetate-polyethylene glycol graft copolymer, and sodium carboxymethyl-cellulose.

The pharmaceutical composition may comprise a processing agent, such as a plasticizer. The pharmaceutical composition may further comprise one or more active pharmaceutical ingredient(s) other than vemurafenib, such as a MEK inhibitor. The pharmaceutical composition may be combined with a co-processed with one or more active pharmaceutical ingredient(s) other than vemurafenib, such as a MEK inhibitor, in a final dosage form. The pharmaceutical composition may be admixed with one or more active pharmaceutical ingredient(s) other than vemurafenib, such as a MEK inhibitor, in a final dosage form.

In addition, novel pharmaceutical compositions or composites made by TKC and discussed above may be further processed according to methods well known to those of skill in the art, including but not limited to hot melt extrusion, melt granulation, compression molding, tablet compression, capsule filling, film-coating, or injection molding into a final product. In certain embodiments, the composite made by TKC is the final product. Another embodiment is directed to addition of vemurafenib and one or more pharmaceutically acceptable excipients in a ratio of about 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10. Yet another embodiment is directed to addition of vemurafenib and one or more pharmaceutically acceptable adjuvants in a ratio of about 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:15, 1:20 1:25, 1:30, 1:40, 1:50, 1:60, 1:70, 1:80, 1:90, 1:100, 1:150, 1:200, 1:300, 1:400 or 1:500. An additional embodiment is directed to addition of vemurafenib and one or more additional active pharmaceutical ingredient (“API”). The ratio of vemurafenib to other API may be 20:1, 16:1, 6:1, and 2:1, including doses of 720 to 960 mg for vemurafenib and 60 to 100 mg cobimetinib once daily.

The thermokinetic processing may be conducted in a thermokinetic chamber. A thermokinetic chamber is an enclosed vessel or chamber in which TKC occurs. In one aspect, the average temperature inside the chamber is ramped up to a pre-defined final temperature over the duration of processing to achieve optimal thermokinetic mixing of vemurafenib and the one or more pharmaceutically acceptable excipients, adjuvants, additional APIs, or any combination thereof, into a composite. In another aspect, multiple speeds are used during a single, rotationally continuous TKC operation to achieve optimal thermokinetic mixing of vemurafenib and one or more pharmaceutically acceptable excipients, adjuvants, additional APIs, or any combination thereof, into a composite with minimal thermal degradation. The length of processing and exposure to elevated temperatures or speeds during thermokinetic mixing will generally be below the thermal sensitivity threshold of vemurafenib, excipient(s), adjuvant(s), or additional API(s). In another aspect, the thermokinetic processing is performed at an average temperature at or below the melting point of vemurafenib, excipient(s), adjuvant(s), or additional API(s); the thermokinetic processing is performed at an average temperature at or below the glass transition temperature of vemurafenib, excipient(s), adjuvant(s), or additional API(s); or the thermokinetic processing is performed at an average temperature at or below the molten transition point of vemurafenib, excipient(s), adjuvant(s), or additional API(s).

In one aspect, the vemurafenib composite made by TKC is a homogenous, heterogenous, or heterogeneously homogenous composite or an amorphous composite. In another aspect, the method, vemurafenib compositions and composite of the present disclosure may be adapted for oral or non-oral administration, for example buccal, sublingual, intravenous, parenteral, pulmonary, rectal, vaginal, topical, urethral, otic, ocular, or transdermal administration. In another aspect, the TKC may be conducted with or without a processing agent. Examples of processing agents include a plasticizer, a thermal lubricant, an organic solvent, an agent that facilitates melt blending, and an agent that facilitates downstream processing (e.g., lecithin). The composite may also include a carrier, e.g., a polymer with a high melt viscosity. In another aspect, the release rate profile of the vemurafenib is determined by the one or more excipients of the composition. As such, the composition may be formulated for immediate release, mixed release, extended release or combinations thereof. In another aspect, the particle size of the vemurafenib is reduced in an excipient/carrier system in which the vemurafenib is not miscible, not compatible, or not miscible or compatible. In one aspect, the vemurafenib is formulated as a nanocomposite with an excipient, a carrier, an adjuvant, or any combination thereof.

In certain embodiments, the thermokinetic processing substantially eliminates vemurafenib, excipient, adjuvant or additional API degradation. For example, TKC may generate compositions and composites with less than about 2.0%, 1.0%, 0.75%, 0.5%, 0.1%, 0.05%, or 0.01% degradation products of vemurafenib, adjuvant, excipient or additional API. This advantage is important for vemurafenib, which is subject to recrystallization during washing and drying during the MBP process. In other embodiments, TKC may generate compositions with a minimum of at least about 90%, 91%, 92%, 93%, 94%, 95%, 96%, 97%, 98%, 99%, or 99.9% drug potency with respect to vemurafenib. Examples of TKC may be performed for less than 5, 10, 15, 20, 25, 30, 35, 40, 45, 50, 55, 60, 75, 100, 120, 150, 180, 240 and 300 seconds. Generally, TKC may be performed for less than 5, 10, 15, 20, 25, 30, 35, 40, 45, 50, 55, 60, 75, 100, 120, 150, 180, 240 and 300 seconds, and any ranges therein. In certain embodiments, the vemurafenib has amorphous, crystalline, or intermediate morphology.

In certain embodiments, the formulations may provide for enhanced solubility of vemurafenib through the mixing of vemurafenib with pharmaceutically acceptable polymers, carriers, surfactants, excipients, adjuvants or any combination thereof. Thus, for example, compositions which display enhanced solubility are comprised of vemurafenib and a surfactant or surfactants, vemurafenib and a pharmaceutical carrier (thermal binder) or carriers, or vemurafenib and a combination of a surfactant and pharmaceutical carrier or surfactants and carriers.

A further embodiment of the present disclosure is a pharmaceutical composition comprising vemurafenib, and one or more pharmaceutically acceptable excipients, adjuvants, additional APIs, or a combination thereof, wherein a peak solubility of the vemurafenib in the composition is greater than about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 20 μg/mL, about 25 μg/mL, about 30 μg/mL, about 35 μg/mL, about 40 μg/mL, 45 μg/mL, about 50 μg/mL or about 60 μg/mL in an aqueous buffer of pH between 4 and 8.

A further embodiment of the present disclosure is a pharmaceutical composition comprising vemurafenib and one or more pharmaceutically acceptable excipients, adjuvants, additional APIs, or a combination thereof, wherein a ratio of peak solubility of vemurafenib in the composition over peak solubility of the reference standard vemurafenib, for example processed using the MBP method, is greater than about 3:1, about 4:1, about 5:1, about 6:1, about 7:1, about 8:1, about 9:1, or about 10:1.

A further embodiment of the present disclosure is a pharmaceutical composition comprising vemurafenib and one or more pharmaceutically acceptable excipients, adjuvants, or additional APIs, wherein Cₘₐₓ of the vemurafenib in the composition and Cₘₐₓ of the reference standard vemurafenib, for example processed using the MBP method, when delivered orally have a ratio that is greater than about 5:1, about 6:1, about 7:1, about 8:1, about 10:1, about 12:1, about 15:1 or about 20:1.

A further embodiment of the present disclosure is a method of formulating a pharmaceutical composition comprising vemurafenib and one or more pharmaceutically acceptable excipients, adjuvants, additional APIs, or any combination thereof, by TKC to increase bioavailability of the vemurafenib, comprising thermokinetic processing of the vemurafenib with the one or more pharmaceutically acceptable excipients, adjuvants, additional APIs, or any combination thereof until melt blended into a composite.

A further embodiment of the present disclosure is a pharmaceutical composition comprising vemurafenib and one or more pharmaceutically acceptable excipients, adjuvants, additional APIs, or any combination thereof, wherein the composition is a homogenous, heterogenous, or heterogeneously homogenous composition in which the glass transition temperature is higher than the glass transition temperature of an identical combination of an identical vemurafenib and pharmaceutically acceptable excipients, adjuvants, additional APIs, or any combination thereof processed using a microprecipitated bulk powder method.

A further embodiment of the present disclosure is a pharmaceutical composition comprising vemurafenib and one or more pharmaceutically acceptable excipients, adjuvants, additional APIs, or any combination thereof, wherein the composition is a homogenous, heterogenous, or heterogeneously homogenous composition which has a single glass transition temperature, wherein an identical combination of an identical vemurafenib and pharmaceutically acceptable excipients, adjuvants, additional APIs, or any combination thereof processed using a MBP method has two or more glass transition temperatures.

A further embodiment of the present disclosure is a pharmaceutical composition comprising vemurafenib and one or more pharmaceutically acceptable excipients, adjuvants, additional APIs, or any combination thereof, processed into a composite, wherein the composite is a homogenous, heterogenous, or heterogeneously homogenous composition which has a less than about 1.0%, about 2%, about 3%, about 4% or about 5%, about 6%, about 7%, about 8%, about 9%, or about 10% degradation products of the vemurafenib.

Still another embodiment includes a method of treating cancer comprising administering to a patient in need of such treatment a therapeutically effective amount of the pharmaceutical composition as described above. The cancer may be a solid tumor, colorectal cancer or skin cancer, melanoma, or metastatic melanoma. The patient may be BRAF V600 mutation-positive.

An additional embodiment includes a method of treating cancer comprising administering to a patient in need of such treatment a therapeutically effective amount of the pharmaceutical formulation as described above. The cancer may be a solid tumor, colorectal cancer or skin cancer, melanoma, or metastatic melanoma. The patient may be BRAF V600 mutation-positive.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Batch compositions. VEM.20141022.01 (“01”) is material identical to Zelboraf® MPB. VEM.20141022.03 (“03”) contains a TKC processing aid, sodium stearyl fumarate.

FIG. 2. Processing parameters for 01 and 03 batches. The maximum temperature is 90° C. below the melting point of vemurafenib. The processing time is brief (7 seconds for left panel, 9 second for right panel). Both permit thermal processing to render vemurafenib amorphous without degrading the polymer.

FIG. 3. X-ray diffraction. The results demonstrate that the TKC processed batches are equivalent to the MBP composition with respect to absence of crystalline vemurafenib. Bottom line is raw vemurafenib; second line from the bottom is the MBP product. The middle line is 01. The second line from the top is 03 “tray”. The top line is 03 “door”.

FIG. 4. Modulated differential scanning calorimetry. The results demonstrate that the TKC processed batches are single-phase amorphous dispersions as indicated by a single glass transition temperature.

FIG. 5A. Polarized light microscopy for 01. Left panel shows tray material at ≦250μ @ 10× magnification. Right panel shows tray material at ≦250μ @ 40× magnification. No trace crystallinity is observed.

FIG. 5B. Polarized light microscopy for 03. Left panel shows tray material at ≦250μ @ 40× magnification. Right panel shows tray material at ≦250μ @ 40× magnification. No trace crystallinity is observed.

FIG. 6A. HPLC analysis of 01. No degradation of vemurafenib can be observed compared to the standard.

FIG. 6B. HPLC analysis of 03. No degradation of vemurafenib can be observed compared to the standard.

FIG. 7. Dissolution and Supersaturation. MBP material and TKC Batch 1 showed dissolution of vemurfaenib into solution followed by decreased supersaturation over time. TKC Batch 3 (containing SSF) demonstrated similar release into solution but without significant loss of supersaturation through 8 hours.

FIG. 8. Batch compositions. Batches 4 and 5 utilized VA64 as the polymer carrier. Batches 6 and 7 utilized HPMC as the polymer carrier. Batches 5 and 7 contained DSS as a surfactant.

FIG. 9. Processing parameters for TKC Batches 4 through 7. The maximum temperature was approximately 90° C. below the melting point of vemurafenib. The processing time was brief, between 4 and 9 seconds. All profiles permitted thermal processing to render vemurafenib amorphous with the polymer carrier.

FIG. 10. Powder X-ray diffraction. The results demonstrated that the TKC processed batches were substantially amorphous and had an absence of crystalline vemurafenib. Top lightest gray line is VEM_20141022_04; second from top light gray line is VEM_20141022_05; second from bottom dark gray line is VEM_20141022_06; and bottom gray line is VEM_20141022_07.

### DETAILED DESCRIPTION OF THE INVENTION

Although making and using various embodiments of the present disclosure are discussed in detail below, it should be appreciated that the present disclosure provides many inventive concepts that may be embodied in a wide variety of contexts. The specific aspects and embodiments discussed herein are merely illustrative of ways to make and use the disclosure, and do not limit the scope of the disclosure.

As discussed below, applicants describe improved vemurafenib compositions and methods for their manufacture. These methods permit thermal processing to produce an amorphous solid dispersion of vemurafenib with high amorphous drug loading. Moreover, the non-solvent nature of the process eliminates the issues associated with solvent-based processes, namely, cost, safety, and environmental waste. It is a simpler, more efficient process than the current MBP approach to making vemurafenib pharmaceuticals, and reduces cost of goods and risk for out-of-spec batches. Finally, MBP is limited to ionic polymers, whereas the TKC methods applied here are not. These methods permit unique compositions of vemurafenib with non-ionic, cross-linked, highly viscous, and thermally labile pharmaceutical polymers with additional advantages in drug manufacture and delivery.

These and other aspects of the disclosure are discussed in detail below.

### I. DEFINITIONS

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration.

With regard to the values or ranges recited herein, the term “about” is intended to capture variations above and below the stated number that may achieve substantially the same results as the stated number. In the present disclosure, each of the variously stated ranges is intended to be continuous so as to include each numerical parameter between the stated minimum and maximum value of each range. For example, a range of about 1 to about 4 includes about 1, 1, about 2, 2, about 3, 3, about 4, and 4. The terminology herein is used to describe specific embodiments of the disclosure, but their usage does not delimit the disclosure, except as outlined in the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “thermokinetic compounding” or “TKC” refers to a method of thermokinetic mixing until melt blended. TKC may also be described as a thermokinetic mixing process or thermokinetic processing in which processing ends at a point sometime prior to agglomeration. The commercial name for this process is “KinetiSol®”.

As used herein, the phrase “a homogenous, heterogenous, or heterogeneously homogenous composite or an amorphous composite” refers to the various compositions that can be made using the TKC method.

As used herein, the term “heterogeneously homogenous composite” refers to a material composition having at least two different materials that are evenly and uniformly distributed throughout the volume.

As used herein, the phrase “reference standard active pharmaceutical ingredient” means the most thermodynamically stable form of the active pharmaceutical ingredient that is currently available.

As used herein, the term “substantial degradation,” in conjunction with the term “vemurafenib” or “additional API(s)” refers to degradation leading to the generation of impurities at levels beyond the threshold that has been qualified by toxicology studies, or beyond the allowable threshold for unknown impurities. See, for example Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006. As used herein, the term “substantial degradation,” in conjunction with the term “excipient” refers to decomposition of the excipient to the extent that the excipient would no longer meet the specifications set forth in an official monograph of an accepted pharmacopeia, e.g., the United States Pharmacopeia.

As used herein, the term “high melt viscosity” refers to melt viscosities greater than 10,000 Pa*s.

As used herein, the term “thermally labile API” refers to an API that degrades at its crystalline melting point, or one that degrades at temperatures below the crystalline melting point when in a non-crystalline (amorphous) form. As used herein, the term “thermolabile polymer” refers to a polymer that degrades at or below about 200° C.

Whether the composition of the present disclosure is a homogenous, heterogenous, or heterogeneously homogenous composition, an amorphous composition or combinations thereof, the TKC processing conditions can produce a composition with a glass transition temperature that is higher than the glass transition temperature of an identical combination of the drug and pharmaceutically acceptable excipients, adjuvants, additional APIs, or any combination thereof, thermally processed or processed using the MBP method, for example either with or without the use of a plasticizer. The TKC processing conditions can also produce a composition with a single glass transition temperature, wherein an identical combination of the identical API and pharmaceutically acceptable excipients, adjuvants, additional APIs, or any combination thereof, processed thermally or processed using the MBP method, has two or more glass transition temperatures. In other embodiments, the pharmaceutical compositions of the present disclosure have a single glass transition temperature that is at least about 20%, 30%, 40%, 50%, 60%, 70%, 80%, or 90% higher than the lowest glass transition temperature of the identical combination processed thermally or processed using the MBP method. Alternatively, the compositions made using thermokinetic processing may generate compositions with a minimum of at least about 90%, 91%, 92%, 93%, 94%, 95%, 96%, 97%, 98%, 99%, or 99.9% therapeutic potency with respect to each drug.

As used herein, the term “significantly higher” in conjunction with glass transition temperatures, refers to compositions that have a glass transition temperature that is at least about 20% higher than the lowest glass transition temperature of the identical formulation thermally processed or processed using the MBP method.

As used herein, the term “thermokinetic chamber” refers to an enclosed vessel or chamber in which the TKC method is used to make the novel compositions of the present disclosure.

As used herein, “thermally processed” or “processed thermally” means that components are processed by hot melt extrusion, melt granulation, compression molding, tablet compression, capsule filling, film-coating, or injection molding.

As used herein, “extrusion” is the well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders. Furthermore, the extrusion can be carried out through melt extrusion. Components of the present disclosure can be melted and extruded with a continuous, solvent free extrusion process, with or without inclusion of additives. Such processes are well-known to skilled practitioners in the art.

As used herein, “spray congealing” is a method that is generally used in changing the structure of materials, to obtain free flowing powders from liquids and to provide pellets. Spray congealing is a process in which a substance of interest is allowed to melt, disperse, or dissolve in a hot melt of other additives, and is then sprayed into an air chamber wherein the temperature is below the melting point of the formulation components, to provide congealed pellets. Such a process is well-known to skilled practitioners in the art.

As used herein, “solvent dehydration” or “spray drying technique” is commonly employed to produce a dry powder from a liquid or slurry by rapidly drying with a hot gas. This is one preferred method of drying many thermally-sensitive materials such as foods and pharmaceuticals. Water or organic solvent based formulations can be spray dried by using inert process gas, such as nitrogen, argon and the like. Such a process is well-known to skilled practitioners in the art.

In certain embodiments, the pharmaceutical formulations of the present disclosure can be processed by the techniques of extrusion, melt extrusion, spray congealing, spray drying or any other conventional technique to provide solid compositions from solution, emulsions suspensions or other mixtures of solids and liquids or liquids and liquids.

As used herein, “bioavailability” is a term meaning the degree to which a drug becomes available to the target tissue after being administered to the body. Poor bioavailability is a significant problem encountered in the development of pharmaceutical compositions, particularly those containing a drug that is not highly soluble. In certain embodiments such as formulations of proteins, the proteins may be water soluble, poorly soluble, not highly soluble, or not soluble. The skilled artisan will recognize that various methodologies may be used to increase the solubility of proteins, e.g., use of different solvents, excipients, carriers, formation of fusion proteins, targeted manipulation of the amino acid sequence, glycosylation, lipidation, degradation, combination with one or more salts and the addition of various salts.

As used herein, the phrase “pharmaceutically acceptable” refers to molecular entities, compositions, materials, excipients, carriers, and the like that do not produce an allergic or similar untoward reaction when administered to humans in general.

As used herein, “poorly soluble” refers to drug having a solubility such that the dose to be administered can be dissolved in 250 ml of aqueous media ranging in pH from 1 to 7.5, a drug with a slow dissolution rate, and a drug with a low equilibrium solubility, for example resulting in decreased bioavailability of the pharmacological effect of the therapeutic drug being delivered.

As used herein, “derivative” refers to chemically modified inhibitors or stimulators that still retain the desired effect or property of the original drug. Such derivatives may be derived by the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means known to those of skill in the art. As used herein, “analogs” include structural equivalents or mimetics.

The solution agent used in the solution can be aqueous such as water, one or more organic solvents, or a combination thereof. When used, the organic solvents can be water miscible or non-water miscible. Suitable organic solvents include but are not limited to ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexanes, heptane, pentane, and combinations thereof.

By “immediate release” is meant a release of an API to an environment over a period of seconds to no more than about 30 minutes once release has begun and release begins within no more than about 2 minutes after administration. An immediate release does not exhibit a significant delay in the release of drug.

By “rapid release” is meant a release of an API to an environment over a period of 1-59 minutes or 0.1 minute to three hours once release has begun and release can begin within a few minutes after administration or after expiration of a delay period (lag time) after administration.

As used herein, the term “extended release” profile assumes the definition as widely recognized in the art of pharmaceutical sciences. An extended release dosage form will release an API at a substantially constant rate over an extended period of time or a substantially constant amount of API will be released incrementally over an extended period of time. An extended release tablet generally effects at least a two-fold reduction in dosing frequency as compared to the API presented in a conventional dosage form (e.g., a solution or rapid releasing conventional solid dosage forms).

By “controlled release” is meant a release of an API to an environment over a period of about eight hours up to about 12 hours, 16 hours, 18 hours, 20 hours, a day, or more than a day. By “sustained release” is meant an extended release of an active agent to maintain a constant drug level in the blood or target tissue of a subject to which the device is administered.

The term “controlled release”, as regards to drug release, includes the terms “extended release,” “prolonged release,” “sustained release,” or “slow release,” as these terms are used in the pharmaceutical sciences. A controlled release can begin within a few minutes after administration or after expiration of a delay period (lag time) after administration.

A “slow release dosage form” is one that provides a slow rate of release of API so that API is released slowly and approximately continuously over a period of 3 hours, 6 hours, 12 hours, 18 hours, a day, 2 or more days, a week, or 2 or more weeks, for example.

The term “mixed release” as used herein refers to a pharmaceutical agent that includes two or more release profiles for one or more active pharmaceutical ingredients. For example, the mixed release may include an immediate release and an extended release portion, each of which may be the same API or each may be a different API.

A “timed release dosage form” is one that begins to release an API after a predetermined period of time as measured from the moment of initial exposure to the environment of use.

A “targeted release dosage form” generally refers to an oral dosage form that is designed to deliver an API to a particular portion of the gastrointestinal tract of a subject. An exemplary targeted dosage form is an enteric dosage form that delivers a drug into the middle to lower intestinal tract but not into the stomach or mouth of the subject. Other targeted dosage forms can deliver to other sections of the gastrointestinal tract such as the stomach, jejunum, ileum, duodenum, cecum, large intestine, small intestine, colon, or rectum.

By “delayed release” is meant that initial release of an API occurs after expiration of an approximate delay (or lag) period. For example, if release of an API from an extended release composition is delayed two hours, then release of the API begins at about two hours after administration of the composition, or dosage form, to a subject. In general, a delayed release is opposite of an immediate release, wherein release of an API begins after no more than a few minutes after administration. Accordingly, the API release profile from a particular composition can be a delayed-extended release or a delayed-rapid release. A “delayed-extended” release profile is one wherein extended release of an API begins after expiration of an initial delay period. A “delayed-rapid” release profile is one wherein rapid release of an API begins after expiration of an initial delay period.

A “pulsatile release dosage form” is one that provides pulses of high API concentration, interspersed with low concentration troughs. A pulsatile profile containing two peaks may be described as “bimodal.” A pulsatile profile of more than two peaks may be described as multi-modal.

A “pseudo-first order release profile” is one that approximates a first order release profile. A first order release profile characterizes the release profile of a dosage form that releases a constant percentage of an initial API charge per unit time.

A “pseudo-zero order release profile” is one that approximates a zero-order release profile. A zero-order release profile characterizes the release profile of a dosage form that releases a constant amount of API per unit time.

### II. THERMOKINETIC COMPOUNDING

In certain embodiments, the pharmaceutical formulations of the present disclosure are processed in a thermokinetic chamber as disclosed in U.S. Pat. No. 8,486,423, which is incorporated herein by reference. This disclosure is directed to a method of blending certain heat sensitive or thermolabile components in a thermokinetic mixer by using multiple speeds during a single, rotationally continuous operation on a batch containing thermolabile components in order to minimize any substantial thermal degradation, so that the resulting pharmaceutical compositions have increased bioavailability and stability.

In a TKC chamber the average temperature inside the chamber is ramped up to a pre-defined final temperature over the duration of processing to achieve thermokinetic compounding of an API and the one or more pharmaceutically acceptable excipients, adjuvants, additional APIs, or combinations thereof, into a composite. The length of processing and exposure to elevated temperatures during thermokinetic compounding will generally be below the thermal sensitivity threshold of the API, the excipients, the adjuvants, the additional APIs, or all of these. Multiple speeds may be used during a single, rotationally continuous TKC operation to achieve optimal thermokinetic mixing of the API and the one or more pharmaceutically acceptable excipients, adjuvants and additional APIs, or combinations thereof, into a composite with minimal thermal degradation. The pre-defined final temperature and speed(s) are selected to reduce the possibility that the API, excipients, adjuvants, additional APIs and/or processing agents are degraded or their functionality is impaired during processing. Generally, the pre-defined final temperature, pressure, time of processing and other environmental conditions (e.g., pH, moisture, buffers, ionic strength, O₂) will be selected to substantially eliminate API, excipient, adjuvant, additional APIs and/or processing agent degradation.

One embodiment is a method for continuous blending and melting of an autoheated mixture in the mixing chamber of a high speed mixer, where a first speed is changed mid-processing to a second speed upon achieving a first desired process parameter. Another embodiment is the use of variations in the shape, width and angle of the facial portions of the shaft extensions or projections that intrude into the main processing volume to control translation of rotational shaft energy delivered to the extensions or projections into heating energy within particles impacting the portions of the extensions or projections. Other embodiments include:
producing solid dispersions of vemurafenib, with or without additional APIs, by processing at low temperatures for very brief durations;
producing solid dispersions of vemurafenib, with or without additional APIs, in thermolabile polymers and excipients by processing at low temperatures for very brief durations;
rendering vemurafenib, with or without additional APIs, amorphous while dispersing in a polymeric, non-polymeric, or combination excipient carrier system;
rendering vemurafenib, with or without additional APIs, amorphous while dispersing in a polymeric, non-polymeric, or combination excipient carrier system and adjuvants;
dry milling of crystalline vemurafenib to reduce the particle size of the bulk material;
wet milling of crystalline vemurafenib with a pharmaceutically acceptable solvent to reduce the particle size of the bulk material;
melt milling of crystalline vemurafenib with one or more molten pharmaceutical excipients having limited miscibility with the crystalline vemurafenib to reduce the particle size of the bulk material;
milling crystalline vemurafenib in the presence of polymeric or non-polymeric excipient to create ordered mixtures where fine vemurafenib particles adhere to the surface of excipient particles and/or excipient particles adhere to the surface of fine vemurafenib particles;
producing single phase, miscible composites of vemurafenib and one or more other pharmaceutical materials previously considered to be immiscible for utilization in a secondary processing step, e.g. melt extrusion, film coating, tableting and granulation;
pre-plasticizing polymeric materials for subsequent use in film coating or melt extrusion operations;
rendering a crystalline or semi-crystalline pharmaceutical polymer amorphous, which can be used as a carrier for vemurafenib, in which the amorphous character improves the dissolution rate of the vemurafenib-polymer composite, the stability of the vemurafenib-polymer composite, and/or the miscibility of the vemurafenib and the polymer;
deaggregating and dispersing engineered particles in a polymeric carrier without altering the properties of the engineered particles;
simple blending of vemurafenib, with or without additional APIs, in powder form with one or more pharmaceutical excipients;
producing composites comprising vemurafenib, with or without additional APIs, and one or more thermolabile polymers without the use of processing agents; and
homogenously dispersing vemurafenib, with or without additional APIs, with a coloring agent or opacifying agent within a polymer carrier or excipient blend.

Additionally, compositions of the present disclosure may be processed using any technique known to one skilled in the art to produce a solid formulation, including fusion or solvent based techniques. Specific examples of these techniques include extrusion, melt extrusion, hot-melt extrusion, spray congealing, spray drying, hot-spin mixing, ultrasonic compaction, and electrostatic spinning.

### III. VEMURAFENIB

A. Background

Vemurafenib (INN, marketed as ZELBORAF®) is a b-raf enzyme inhibitor developed by Plexxikon and Genentech for the treatment of late-stage melanoma. The name “vemurafenib” comes from V600E mutated BRAF inhibition. The structure is shown below:

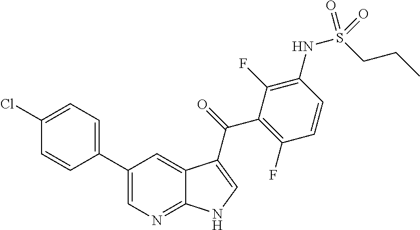 [SMILES: CCCS(=O)(=O)Nc1ccc(F)c(C(=O)c2c[nH]c3ncc(-c4ccc(Cl)cc4)cc23)c1F]

Vemurafenib received FDA approval for the treatment of late-stage melanoma in 2011, making it the first drug designed using fragment-based lead discovery to gain regulatory approval. Vemurafenib later received Health Canada and European approval in 2012 as a monotherapy for the treatment of adult patients with BRAF V600 mutation positive unresectable or metastatic melanoma, the most aggressive form of skin cancer.

Vemurafenib has been shown to cause programmed cell death in melanoma cell lines. Vemurafenib interrupts the b-raf/MEK step on the b-raf/MEK/ERK pathway if the b-raf has the common V600E mutation. About 60% of melanomas have this mutation. It also has efficacy against the rarer BRAF V600K mutation. Melanoma cells without these mutations are not inhibited by vemurafenib; the drug paradoxically stimulates normal BRAF and may promote tumor growth in such cases.

Three mechanisms of resistance to vemurafenib (covering 40% of cases) have been discovered. First, the cancer cells can begin to overexpress a cell surface protein PDGFRB creating an alternative survival pathway. Second, an oncogene called n-ras mutates, reactivating the normal BRAF survival pathway. And third, stromal cells may secrete hepatocyte growth factor (HGF).

In a phase I clinical study, vemurafenib (then known as PLX4032) was able to reduce numbers of cancer cells in over half of a group of 16 patients with advanced melanoma, and the treated group had a median increased survival time of 6 months over the control group. A second phase I study, in patients with a V600E mutation in b-raf, ˜80% showed partial to complete regression. However the regression only lasted from 2 to 18 months.

In early 2010, a Phase I trial for solid tumors (including colorectal cancer), and a phase II study (for metastatic melanoma) were ongoing, and a phase III trial (versus dacarbazine) in patients with previously untreated metastatic melanoma had been started. In June 2011, positive results were reported from the phase III BRIM3 BRAF-mutation melanoma study. Further trials are planned including a trial where vemurafenib will be co-administered with GDC-0973, a MEK-inhibitor The BRIM3 trial reported good updated results in 2012.

At the maximum tolerated dose (MTD) of 960 mg twice a day 31% of patients get skin lesions that may need surgical removal. The BRIM-2 trial investigated 132 patients; the most common adverse events were arthralgia in 58% of patients, skin rash in 52%, and photosensitivity in 52%. In order to better manage side effects some form of dose modification was necessary in 45% of patients. The median daily dose was 1750 mg, which is 91% of the MTD.

Vemurafenib tablets contain 240 mg of vemurafenib as a co-precipitate of vemurafenib and hypromellose acetate succinate (HPMCAS). U.S. Pat. No. 7,863,288 discloses vemurafenib. WO 2010/114928 discloses crystalline forms I and II of vemurafenib; its mesylate, tosylate, maleate, oxalate, dichloroacetate salts, as well as solid dispersions that include vemurafenib and a ionic polymer, in a ratio of vemurafenib and the ionic polymer of about 1:9 to about 5:5, preferably about 3:7 (by weight). WO 2010/129570 discloses non-crystalline complexes of vemurafenib and its L-arginine and L-lysine salts. WO 2011/057974 describes a solid dispersion of vemurafenib, and describes that the amorphous form of vemurafenib has improved solubility in water as compared to the crystalline form, but it is unstable. WO 2012/161776 discloses additional solid forms and salts of vemurafenib, including a hydrochloride salt. WO 2014/008270 discloses vemurafenib choline salts and solid state forms thereof. Thus, “vemurafenib” as used herein may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

B. Melanoma

Melanoma is less common than other skin cancers. However, it is much more dangerous if it is not found early. It causes the majority (75%) of deaths related to skin cancer. Worldwide, doctors diagnose about 160,000 new cases of melanoma yearly. It is more common in women than in men. In women, the most common site is the legs and melanomas in men are most common on the back. It is particularly common among Caucasians, especially northern Europeans living in sunny climates. There are high rates of incidence in Australia, New Zealand, North America (especially Texas and Florida), Latin America, and Northern Europe, with a paradoxical decrease in southern Italy and Sicily. This geographic pattern reflects the primary cause, ultraviolet light (UV) exposure crossed with the amount of skin pigmentation in the population.

1. Early Signs

Early signs of melanoma are changes to the shape or color of existing moles or, in the case of nodular melanoma, the appearance of a new lump anywhere on the skin (such lesions should be referred without delay to a dermatologist). At later stages, the mole may itch, ulcerate or bleed. Early signs of melanoma are summarized by the mnemonic “ABCDE”: Asymmetry, Borders (irregular), Color (variegated), Diameter (greater than 6 mm (0.24 in), about the size of a pencil eraser) and Evolving over time. These classifications do not, however, apply to the most dangerous form of melanoma, nodular melanoma, which has its own classifications: Elevated above the skin surface, Firm to the touch and Growing.

Metastatic melanoma may cause nonspecific paraneoplastic symptoms, including loss of appetite, nausea, vomiting and fatigue. Metastasis of early melanoma is possible, but relatively rare: less than a fifth of melanomas diagnosed early become metastatic. Brain metastases are particularly common in patients with metastatic melanoma. It can also spread to the liver, bones, abdomen or distant lymph nodes.

2. Development

The earliest stage of melanoma starts when the melanocytes begin to grow out of control. Melanocytes are found between the outer layer of the skin (the epidermis) and the next layer (the dermis). This early stage of the disease is called the radial growth phase, and the tumor is less than 1 mm thick. Because the cancer cells have not yet reached the blood vessels lower down in the skin, it is very unlikely that this early-stage cancer will spread to other parts of the body. If the melanoma is detected at this stage, then it can usually be completely removed with surgery. When the tumor cells start to move in a different direction—vertically up into the epidermis and into the papillary dermis—the behavior of the cells changes dramatically.

The next step in the evolution is the invasive radial growth phase, which is a confusing term; however, it explains the next step in the process of the radial growth, when individual cells start to acquire invasive potential. This step is important—from this point on the melanoma is capable of spreading. The Breslow's depth of the lesion is usually less than 1 mm (0.04 in), the Clark level is usually 2.

The following step in the process is the invasive melanoma—the vertical growth phase (VGP). The tumor attains invasive potential, meaning it can grow into the surrounding tissue and can spread around the body through blood or lymph vessels. The tumor thickness is usually more than 1 mm (0.04 in), and the tumor involves the deeper parts of the dermis. The host elicits an immunological reaction against the tumor (during the VGP), which is judged by the presence and activity of the tumor infiltrating lymphocytes (TILs). These cells sometimes completely destroy the primary tumor; this is called regression, which is the latest stage of the melanoma development. In certain cases, the primary tumor is completely destroyed and only the metastatic tumor is discovered.

3. Detection

Visual diagnosis of melanomas is still the most common method employed by health professionals. Moles that are irregular in color or shape are often treated as candidates of melanoma. The diagnosis of melanoma requires experience, as early stages may look identical to harmless moles or not have any color at all. People with a personal or family history of skin cancer or of dysplastic nevus syndrome (multiple atypical moles) should see a dermatologist at least once a year to be sure they are not developing melanoma. There is no blood test for detecting melanomas.

Many melanomas present themselves as lesions smaller than 6 mm in diameter; and all melanomas were malignant on day 1 of growth, which is merely a dot. An astute physician will examine all abnormal moles, including ones less than 6 mm in diameter. Seborrheic keratosis may meet some or all of the ABCD criteria, and can lead to false alarms among laypeople and sometimes even physicians. An experienced doctor can generally distinguish seborrheic keratosis from melanoma upon examination, or with dermatoscopy.

Total body photography, which involves photographic documentation of as much body surface as possible, is often used during follow-up of high-risk patients. The technique has been reported to enable early detection and provides a cost-effective approach (being possible with the use of any digital camera), but its efficacy has been questioned due to its inability to detect macroscopic changes. The diagnosis method should be used in conjunction with (and not as a replacement for) dermoscopic imaging, with a combination of both methods appearing to give extremely high rates of detection.

Melanoma is divided into the following types: lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a spitz nevus, and uveal melanoma.

Confirmation of the clinical diagnosis is achieved with a skin biopsy. This is usually followed up with a wider excision of the scar or tumor. Depending on the stage, a sentinel lymph node biopsy is done, as well, although controversy exists around trial evidence for this procedure. Treatment of advanced malignant melanoma is performed from a multidisciplinary approach.

4. Staging

Melanoma stages are listed below with their 5 year survival rates:

Stage 0: Melanoma in situ (Clark Level I), 99.9% survival

Stage I/II: Invasive melanoma, 85-99% survival
T1a: Less than 1.00 mm primary tumor thickness, without ulceration, and mitosis ≦1/mm²
T1b: Less than 1.00 mm primary tumor thickness, with ulceration or mitoses ≧1/mm²
T2a: 1.00-2.00 mm primary tumor thickness, without ulceration

Stage II: High risk melanoma, 40-85% survival
T2b: 1.00-2.00 mm primary tumor thickness, with ulceration
T3a: 2.00-4.00 mm primary tumor thickness, without ulceration
T3b: 2.00-4.00 mm primary tumor thickness, with ulceration
T4a: 4.00 mm or greater primary tumor thickness without ulceration
T4b: 4.00 mm or greater primary tumor thickness with ulceration

Stage III: Regional metastasis, 25-60% survival
N1: Single positive lymph node
N2: Two to three positive lymph nodes or regional skin/in-transit metastasis
N3: Four positive lymph nodes or one lymph node and regional skin/in-transit metastases

Stage IV: Distant metastasis, 9-15% survival
M1a: Distant skin metastasis, normal LDH
M1b: Lung metastasis, normal LDH
M1c: Other distant metastasis or any distant metastasis with elevated LDH

5. Prognosis

Features that affect prognosis are tumor thickness in millimeters (Breslow's depth), depth related to skin structures (Clark level), type of melanoma, presence of ulceration, presence of lymphatic/perineural invasion, presence of tumor-infiltrating lymphocytes (if present, prognosis is better), location of lesion, presence of satellite lesions, and presence of regional or distant metastasis. Certain types of melanoma have worse prognoses but this is explained by their thickness. Interestingly, less invasive melanomas even with lymph node metastases carry a better prognosis than deep melanomas without regional metastasis at time of staging. Local recurrences tend to behave similarly to a primary unless they are at the site of a wide local excision (as opposed to a staged excision or punch/shave excision) since these recurrences tend to indicate lymphatic invasion.

When melanomas have spread to the lymph nodes, one of the most important factors is the number of nodes with malignancy. Extent of malignancy within a node is also important; micrometastases in which malignancy is only microscopic have a more favorable prognosis than macrometastases. In some cases micrometastases may only be detected by special staining, and if malignancy is only detectable by a rarely employed test known as the polymerase chain reaction (PCR), the prognosis is better. Macrometastases in which malignancy is clinically apparent (in some cases cancer completely replaces a node) have a far worse prognosis, and if nodes are matted or if there is extracapsular extension, the prognosis is still worse.

When there is distant metastasis, the cancer is generally considered incurable. The five year survival rate is less than 10%. The median survival is 6 to 12 months. Treatment is palliative, focusing on life-extension and quality of life. In some cases, patients may live many months or even years with metastatic melanoma (depending on the aggressiveness of the treatment). Metastases to skin and lungs have a better prognosis. Metastases to brain, bone and liver are associated with a worse prognosis.

There is not enough definitive evidence to adequately stage, and thus give a prognosis for ocular melanoma and melanoma of soft parts, or mucosal melanoma (e.g., rectal melanoma), although these tend to metastasize more easily. Even though regression may increase survival, when a melanoma has regressed, it is impossible to know its original size and thus the original tumor is often worse than a pathology report might indicate.

6. Treatment

Excisional biopsies may remove the tumor, but further surgery is often necessary to reduce the risk of recurrence. Complete surgical excision with adequate surgical margins and assessment for the presence of detectable metastatic disease along with short- and long-term follow-up is standard. Often this is done by a wide local excision (WLE) with 1 to 2 cm margins. Melanoma-in-situ and lentigo malignas are treated with narrower surgical margins, usually 0.2 to 0.5 cm. Many surgeons consider 0.5 cm the standard of care for standard excision of melanoma-in-situ, but 0.2 cm margin might be acceptable for margin controlled surgery (Mohs surgery, or the double-bladed technique with margin control). The wide excision aims to reduce the rate of tumor recurrence at the site of the original lesion. This is a common pattern of treatment failure in melanoma. Considerable research has aimed to elucidate appropriate margins for excision with a general trend toward less aggressive treatment during the last decades.

Mohs surgery has been reported with cure rate as low as 77% and as high as 98% for melanoma-in-situ. CCPDMA and the “double scalpel” peripheral margin controlled surgery is equivalent to Mohs surgery in effectiveness on this “intra-epithelial” type of melanoma.

Melanomas that spread usually do so to the lymph nodes in the area of the tumor before spreading elsewhere. Attempts to improve survival by removing lymph nodes surgically (lymphadenectomy) were associated with many complications, but no overall survival benefit. Recently, the technique of sentinel lymph node biopsy has been developed to reduce the complications of lymph node surgery while allowing assessment of the involvement of nodes with tumor.

Although controversial and without prolonging survival, sentinel lymph node biopsy is often performed, especially for T1b/T2+ tumors, mucosal tumors, ocular melanoma and tumors of the limbs. A process called lymphoscintigraphy is performed in which a radioactive tracer is injected at the tumor site to localize the sentinel node(s). Further precision is provided using a blue tracer dye, and surgery is performed to biopsy the node(s). Routine hematoxylin and eosin (H&E) and immunoperoxidase staining will be adequate to rule out node involvement. Polymerase chain reaction (PCR) tests on nodes, usually performed to test for entry into clinical trials, now demonstrate that many patients with a negative sentinel lymph node actually had a small number of positive cells in their nodes. Alternatively, a fine-needle aspiration biopsy may be performed and is often used to test masses.

If a lymph node is positive, depending on the extent of lymph node spread, a radical lymph node dissection will often be performed. If the disease is completely resected, the patient will be considered for adjuvant therapy. Excisional skin biopsy is the management of choice. Here, the suspect lesion is totally removed with an adequate (but minimal, usually 1 or 2 mm) ellipse of surrounding skin and tissue. To avoid disruption of the local lymphatic drainage, the preferred surgical margin for the initial biopsy should be narrow (1 mm). The biopsy should include the epidermal, dermal, and subcutaneous layers of the skin. This enables the histopathologist to determine the thickness of the melanoma by microscopic examination. This is described by Breslow's thickness (measured in millimeters). However, for large lesions, such as suspected lentigo maligna, or for lesions in surgically difficult areas (face, toes, fingers, eyelids), a small punch biopsy in representative areas will give adequate information and will not disrupt the final staging or depth determination. In no circumstances should the initial biopsy include the final surgical margin (0.5 cm, 1.0 cm, or 2 cm), as a misdiagnosis can result in excessive scarring and morbidity from the procedure. A large initial excision will disrupt the local lymphatic drainage and can affect further lymphangiogram-directed lymph node dissection. A small punch biopsy can be used at any time where for logistical and personal reasons a patient refuses more invasive excisional biopsy. Small punch biopsies are minimally invasive and heal quickly, usually without noticeable scarring.

High-risk melanomas may require adjuvant treatment, although attitudes to this vary in different countries. In the United States, most patients in otherwise good health will begin up to a year of high-dose interferon treatment, which has severe side effects, but may improve the patient's prognosis slightly. However British Association of Dermatologist guidelines on melanoma state that interferon is not recommended as a standard adjuvant treatment for melanoma. A 2011 meta-analysis showed that interferon could lengthen the time before a melanoma comes back but increased survival by only 3% at 5 years. The unpleasant side effects also greatly decrease quality of life. In Europe, interferon is usually not used outside the scope of clinical trials.

Metastatic melanomas can be detected by X-rays, CT scans, MRIs, PET and PET/CTs, ultrasound, LDH testing and photoacoustic detection. Various chemotherapy agents also are used, including dacarbazine (also termed DTIC), immunotherapy (with interleukin-2 (IL-2) or interferon (IFN)), as well as local perfusion, are used by different centers. The overall success in metastatic melanoma is quite limited. IL-2 (Proleukin) is the first new therapy approved for the treatment of metastatic melanoma in 20 years. Studies have demonstrated that IL-2 offers the possibility of a complete and long-lasting remission in this disease, although only in a small percentage of patients. A number of new agents and novel approaches are under evaluation and show promise. Clinical trial participation should be considered the standard of care for metastatic melanoma.

For lentigo maligna treatment, standard excision is still being performed by most surgeons. Unfortunately, the recurrence rate is exceedingly high (up to 50%). This is due to the ill-defined visible surgical margin, and the facial location of the lesions (often forcing the surgeon to use a narrow surgical margin). The narrow surgical margin used, combined with the limitation of the standard “bread-loafing” technique of fixed tissue histology, result in a high “false negative” error rate, and frequent recurrences. Margin control (peripheral margins) is necessary to eliminate the false negative errors. If bread loafing is used, distances from sections should approach 0.1 mm to assure that the method approaches complete margin control.

Some melanocytic nevi and melanoma-in-situ (lentigo maligna) have resolved with an experimental treatment: imiquimod (Aldara) topical cream, an immune enhancing agent. Some dermasurgeons are combining the 2 methods: surgically excising the cancer and then treating the area with Aldara cream postoperatively for three months.

Radiation therapy is often used after surgical resection for patients with locally or regionally advanced melanoma or for patients with unresectable distant metastases. It may reduce the rate of local recurrence but does not prolong survival. Radioimmunotherapy of metastatic melanoma is currently under investigation. Radiotherapy has a role in the palliation of metastatic melanoma.

C. Delivery

A variety of administration routes are available for delivering vemurafenib to a patient in need. The particular route selected will depend upon the particular drug selected, the weight and age of the patient, and the dosage required for therapeutic effect. The pharmaceutical compositions may conveniently be presented in unit dosage form. Vemurafenib suitable for use in accordance with the present disclosure, and its pharmaceutically acceptable salts, derivatives, analogs, prodrugs, and solvates thereof, can be administered alone, but will generally be administered in admixture with a suitable pharmaceutical excipient, adjuvant, diluent, or carrier selected with regard to the intended route of administration and standard pharmaceutical practice, and can in certain instances be administered with one or more additional API(s), preferably in the same unit dosage form.

Vemurafenib may be used in a variety of application modalities, including oral delivery as tablets, capsules or suspensions; pulmonary and nasal delivery; topical delivery as emulsions, ointments or creams; transdermal delivery; and parenteral delivery as suspensions, microemulsions or depot. As used herein, the term “parenteral” includes subcutaneous, intravenous, intramuscular, or infusion routes of administration.

D. Excipients

The excipients and adjuvants that may be used in the presently disclosed compositions and composites, while potentially having some activity in their own right, for example, antioxidants, are generally defined for this application as compounds that enhance the efficiency and/or efficacy of vemurafenib. It is also possible to have more than one API in a given solution, so that the particles formed contain more than one API.

Any pharmaceutically acceptable excipient known to those of skill in the art may be used to produce the composites and compositions disclosed herein. Examples of excipients for use with the present invention include, but are not limited to, e.g., a pharmaceutically acceptable polymer, a thermolabile polymeric excipient, or a non-polymeric excipient. Other non-limiting examples of excipients include, lactose, glucose, starch, calcium carbonate, kaoline, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used to enhance the efficacy and efficiency of the API. Additional non-limiting examples of compounds that can be included are binders, carriers, cryoprotectants, lyoprotectants, surfactants, fillers, stabilizers, polymers, protease inhibitors, antioxidants, bioavailability enhancers and absorption enhancers. The excipients may be chosen to modify the intended function of the active ingredient by improving flow, or bio-availability, or to control or delay the release of the API. Specific nonlimiting examples include: sucrose, trehaolose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphadityl choline, glycolic acid and salts, deoxycholic acid and salts, sodium fusidate, cyclodextrins, polyethylene glycols, Labrasol®, polyvinyl alcohols, polyvinyl pyrrolidones and tyloxapol. Using the process of the present disclosure, the morphology of the active ingredients can be modified, resulting in highly porous microparticles and nanoparticles.

Exemplary polymer carriers or thermal binders that may be used in the presently disclosed compositions and composites include but are not limited to polyethylene oxide; polypropylene oxide; polyvinylpyrrolidone; polyvinylpyrrolidone-co-vinylacetate; acrylate and methacrylate copolymers; polyethylene; polycaprolactone; polyethylene-co-polypropylene; alkylcelluloses such as methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; polysaccharides such as tragacanth, gum arabic, guar gum, and xanthan gum. One embodiment of the binder is poly(ethylene oxide) (PEO), which can be purchased commercially from companies such as the Dow Chemical Company, which markets PEO under the POLY OX® exemplary grades of which can include WSR N80 having an average molecular weight of about 200,000; 1,000,000; and 2,000,000.

Suitable polymer carriers or thermal binders that may or may not require a plasticizer include, for example, Eudragit® RS PO, Eudragit® 5100, Kollidon® SR (poly(vinyl acetate)-co-poly(vinylpyrrolidone) copolymer), Ethocel® (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), sodium carboxymethyl-cellulose (CMC), dimethylaminoethyl methacrylate-methacrylic acid ester copolymer, ethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), polyvinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit® L-30-D (MA-EA, 1:1), Eudragit® L100-55 (MA-EA, 1:1), Eudragit® EPO (poly(butyl methacylate-co-(2-dimethylaminoethyl) methacrylate-co-methyl methacrylate) 1:2:1), hydroxypropylmethylcellulose acetate succinate (HPMCAS), Coateric® (PVAP), Aquateric® (CAP), and AQUACOAT® (HPMCAS), Soluplus® (polyvinyl caprolactam-polyvinyl acetate-polyethylene glycol graft copolymer, BASF), Luvitec® K 30 (polyvinylpyrrolidone, PVP), Kollidon® (polyvinylpyrrolidone, PVP), polycaprolactone, starches, pectins; polysaccharides such as tragacanth, gum arabic, guar gum, and xanthan gum.

The stabilizing and non-solubilizing carrier may also contain various functional excipients, such as: hydrophilic polymer, antioxidant, super-disintegrant, surfactant including amphiphilic molecules, wetting agent, stabilizing agent, retardant, similar functional excipient, or combination thereof, and plasticizers including citrate esters, polyethylene glycols, PG, triacetin, diethylphthalate, castor oil, and others known to those or ordinary skill in the art. Extruded material may also include an acidifying agent, adsorbent, alkalizing agent, buffering agent, colorant, flavorant, sweetening agent, diluent, opaquant, complexing agent, fragrance, preservative or a combination thereof.

Exemplary hydrophilic polymers which can be a primary or secondary polymeric carrier that can be included in the composites or composition disclosed herein include polyvinyl alcohol) (PVA), polyethylene-polypropylene glycol (e.g., POLOXAMER®), carbomer, polycarbophil, or chitosan. Hydrophilic polymers for use with the present disclosure may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

Compositions with enhanced solubility may comprise a mixture of vemurafenib and an additive that enhances the solubility of the vemurafenib. Examples of such additives include but are not limited to surfactants, polymer carriers, pharmaceutical carriers, thermal binders or other excipients. A particular example may be a mixture of vemurafenib with a surfactant or surfactants, vemurafenib with a polymer or polymers, or vemurafenib with a combination of a surfactant and polymer carrier or surfactants and polymer carriers. A further example is a composition where the vemurafenib is a derivative or analog thereof.

Surfactants that can be used in the disclosed compositions to enhance solubility have been previously presented. Particular examples of such surfactants include but are not limited to sodium dodecyl sulfate, dioctyl docusate sodium, Tween 80, Span 20, Cremophor® EL or Vitamin E TPGS. Polymer carriers that can be used in the disclosed composition to enhance solubility have been previously presented. Particular examples of such polymer carriers include but are not limited to Soluplus®, Eudragit® L100-55, Eudragit® EPO, Kollidon® VA 64, Luvitec®. K 30, Kollidon®, AQOAT®-HF, and AQOAT®-LF. The composition of the present disclosure can thus be any combination of one or more of the APIs, zero, one or more of surfactants or zero, one or more of polymers presented herein.

Solubility can be indicated by peak solubility, which is the highest concentration reached of a species of interest over time during a solubility experiment conducted in a specified medium. The enhanced solubility can be represented as the ratio of peak solubility of the agent in a pharmaceutical composition of the present disclosure compared to peak solubility of the reference standard agent under the same conditions. Preferable, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of vemurafenib that enhance bioavailability may comprise a mixture of vemurafenib and one or more pharmaceutically acceptable adjuvants that enhance the bioavailability of the vemurafenib. Examples of such adjuvants include but are not limited to enzymes inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ of vemurafenib as determined during in vivo testing, where Cₘₐₓ is the highest reached blood level concentration of the vemurafenib over time of monitoring. Enhanced bioavailability can be represented as the ratio of Cₘₐₓ of the vemurafenib in a pharmaceutical composition of the present disclosure compared to Cₘₐₓ of the reference standard vemurafenib under the same conditions. This Cₘₐₓ ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

E. Other API's

MEK is a dual-specificity kinase that phosphorylates the tyrosine and threonine residues on ERKs 1 and 2 required for activation. MEKs are substrates for several protein kinases including the Rafs (c-, A- and B-), Mos, Tp1-2, and MEKK1. MEKs are phosphorylated by these kinases at two serine residues (218 and 222 in rat MEK1). Introduction of acidic residues and truncation of an alpha-helical region in the N-terminal domain causes constitutive activation of MEK. Such proteins are transforming. As such, MEK inhibitors of MEK1 and/or MEK2 can be used to inhibit the MAPK/ERK pathway which is often overactive in some cancers, such as melanoma. Hence MEK inhibitors have potential for treatment of some cancers, especially BRAF-mutated melanoma, and KRAS/BRAF mutated colorectal cancer. One MEK inhibitor is Trametinib (GSK1120212), which is FDA-approved to treat BRAF-mutated melanoma. It is also studied in combination with BRAF inhibitor dabrafenib to treat BRAF-mutated melanoma. Thus, formulations of the instant application may, in addition to vemurafenib, include a MEK inhibitor.

The Phase II Trametinib trial (NCT01245062) was conducted in patients with BRAF_{V600E/K} mutant advanced or malignant melanoma. Patients were randomized 2:1 to Trametinib (2 mg QD) or chemotherapy (dacarbazine or paclitaxel). Patients were stratified by baseline LDH level and prior chemotherapy; patients in the chemotherapy arm were allowed to crossover to receive Trametinib after confirmation of progressive disease. Primary endpoint was progression free survival in patients with BRAF_{V600E} mutation-positive malignant melanoma and no prior brain metastasis; secondary endpoints were overall survival, overall response rate and safety in primary and intention to treat groups. Progression free survival and overall survival were compared using a stratified log-rank test. The study was designed with ≧99% power and one-sided α=0.025 to detect 57% reduction in the risk of progressive disease or death in patients treated with Trametinib versus chemotherapy.

Between December of 2010 and July of 2011, 322 patients were randomized to Trametinib (n=214) or chemotherapy (n=108); 273 patients were BRAF_{V600E} mutation-positive with no prior brain metastasis. HR for primary population for progression free survival by investigator was 0.44 (95% CI 0.31-0.64; p<0.0001) in favor of Trametinib with a median progression free survival of 4.8 mo vs 1.4 mo with chemotherapy. Progression free survival benefit in favor of Trametinib was observed in the intention to treat group; this was confirmed by an independent review. The confirmed overall response rate was 24% with Trametinib and 7% with chemotherapy. HR for interim overall survival was 0.53 (95% CI 0.30-0.94; p=0.0181), in favor of Trametinib in primary population. Overall survival benefit was consistent in the intention to treat population despite 51 patients crossover from chemotherapy to Trametinib. The most frequent adverse events with Trametinib were skin rash, diarrhea, edema, hypertension, fatigue. Known MEKi class effects were observed, e.g., chorioretinopathy (<1%) and decreased ejection fraction (7%). Grade 3 adverse effects in the Trametinib arm were hypertenstion (12%) and rash (7%). The study concluded that Trametinib was the first in class MEKi associated with a significant improvement of progression free survival and overall survival compared to chemotherapy in pts with BRAF_{V600E/K} mutant malignant melanoma.

Others MEK inhibitors include selumetinib, which had a phase 2 clinical trial for non-small cell lung cancer (NSCLC), MEK162, which had a phase 1 trial for biliary tract cancer and melanoma, PD-325901, indicated for breast cancer, colon cancer, and melanoma, as well as XL518, CI-1040, AS703026 (Pimasertib, MSC1936369B), AZD8330(ARRY-424704), Selumetinib (AZD6244), PD035901, Binimetinib, MEK162, PD-325901, Cobimetinib XL518, CI-1040, PD035901 and dabrafinib (Tafinlar®).

### IV. EXAMPLES

It will be understood that particular embodiments described herein are shown by way of illustration and not as limitations of the invention. The principal features of this invention can be employed in various embodiments without departing from the scope of the invention. All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### Example 1

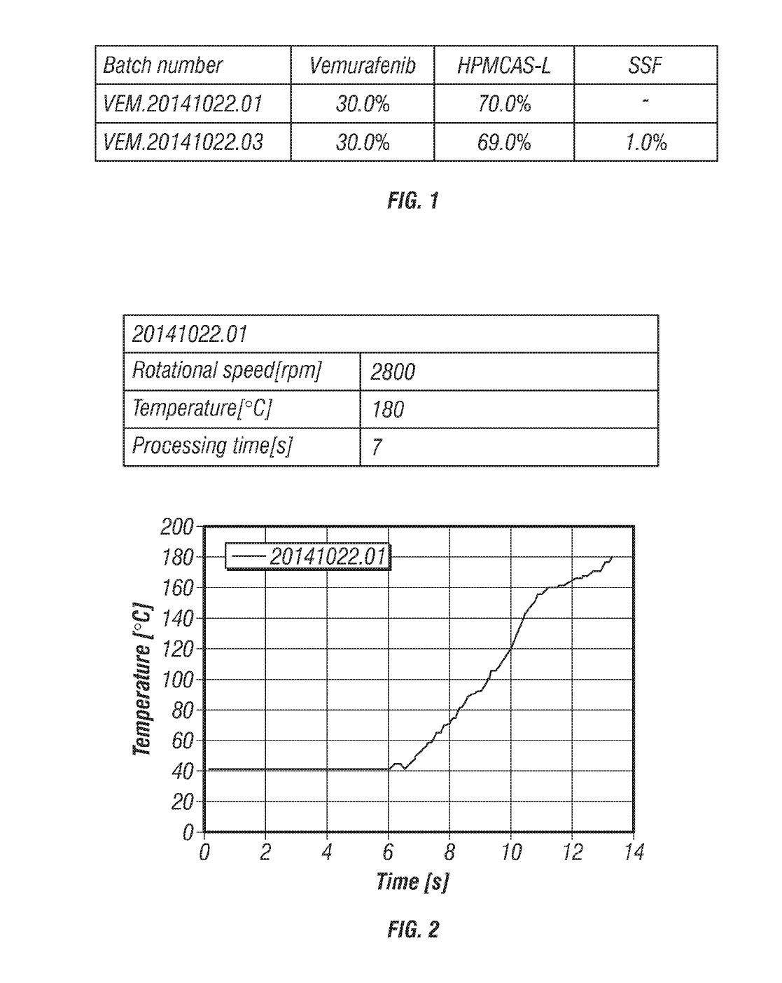

Thermokinetic compounding was performed on the compositions provided in FIG. 1. Batch VEM.20141022.01 (Batch 1) is equivalent in composition to the MBP composition contained in the Zelboraf product. Batch VEM.20141022.03 (Batch 3) contains the addition of 1% sodium stearyl fumarate (SSF) as a lubricant.

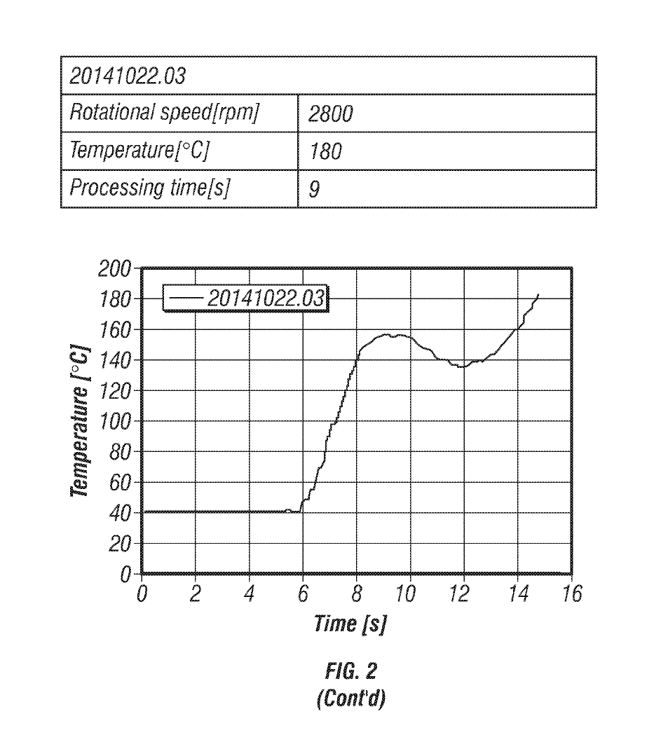

The processing parameters and temperature versus time profiles for TKC processing of Batches 1 and 3 are provided in FIG. 2. This figure signifies that the target amorphous dispersion was achieved by TKC at a peak temperature approximately 90° C. below the melting point of the API and with a time at elevated temperature of less than 10 seconds. Both the low temperature and brief processing duration are critical to producing the amorphous dispersion without degradation to the drug and/or polymer.

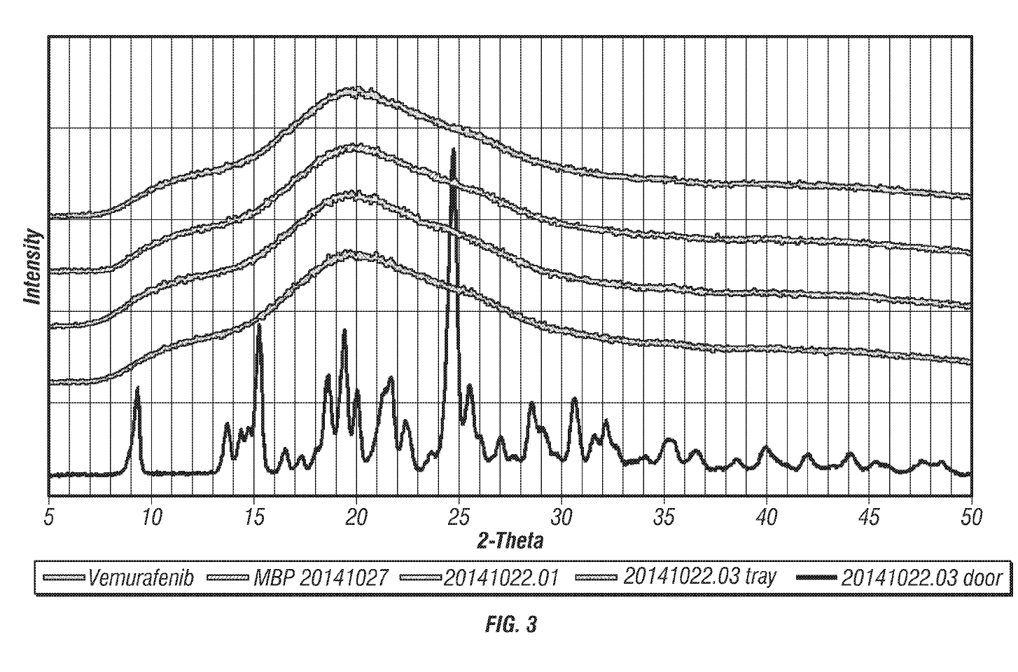

TKC Batches 1 and 3 processed according to the parameters shown in FIG. 2, were analyzed for crystalline content by powder x-ray diffraction (PXRD). The results of the analysis are provided in FIG. 3. These results demonstrate that Bathes 1 and 3 were rendered entirely amorphous by the process (absence of drug-related crystallinity) and are equivalently amorphous to the same composition as produced by MBP.

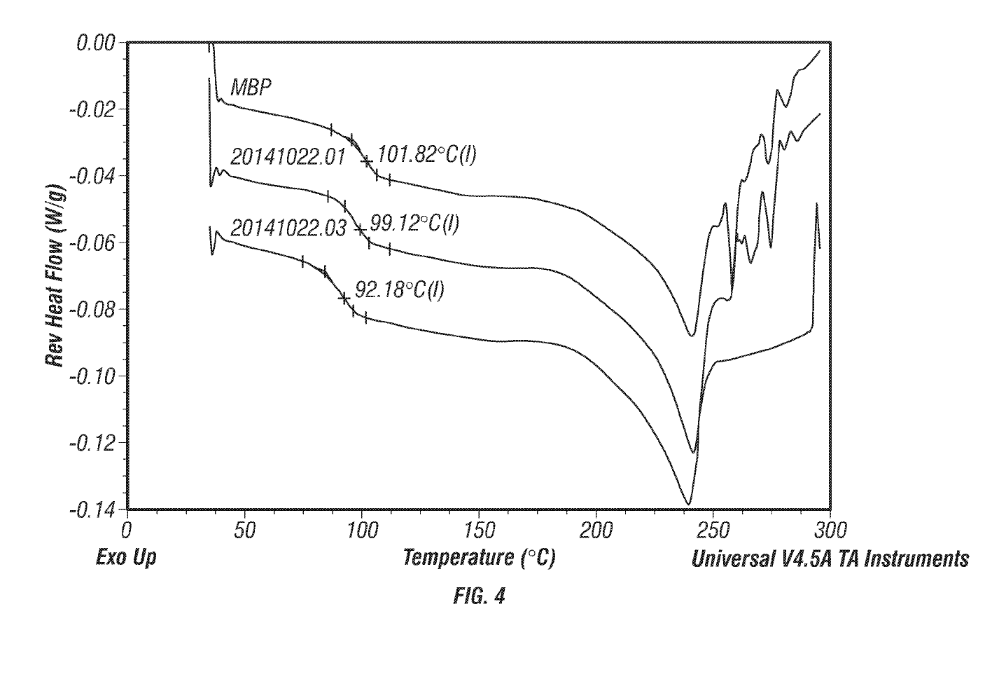

TKC Batches 1 and 3 were also analyzed by modulated differential scanning calorimetry (mDSC) to investigate the nature of the dispersed drug phase in the polymer matrix; a single glass transition temperature by this analysis would represent the most desired single-phase amorphous dispersion. The results of mDSC analysis (FIG. 4) illustrate that both Batch 1 and 3 are single-phase amorphous dispersions as indicated by their single glass transition temperatures with midpoints at 99.12° C. and 92.18° C., respectively. The results are similar to the MBP which was shown to be single phase with a glass transition temperature of 101.82° C.

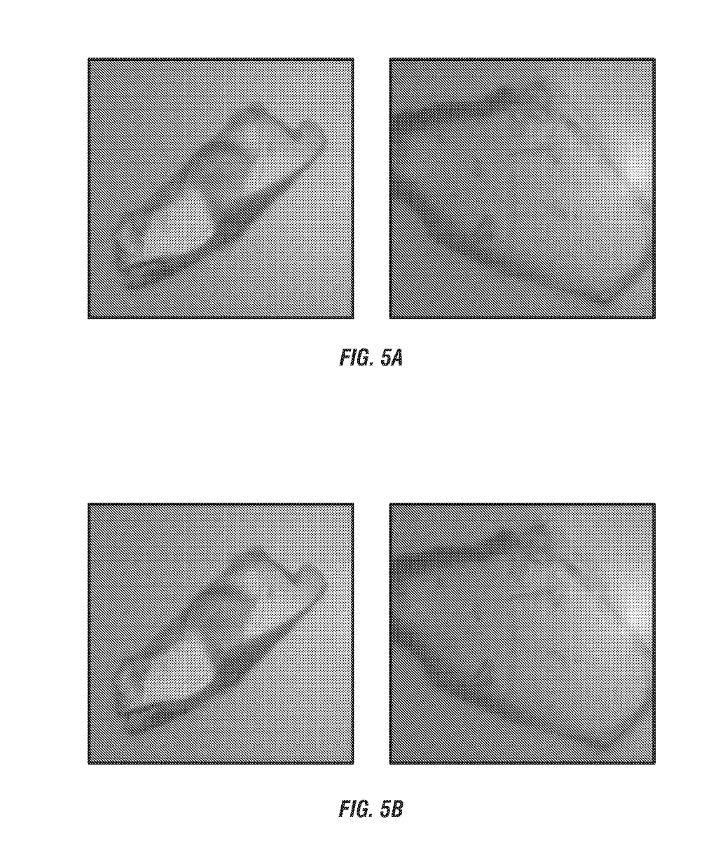

Polarizing light microscopy (PLM) was performed on Batches 1 and 3 to evaluate the possible presence of trace vemurafenib crystals that would not be detected by PXRD and that would be detrimental to the dissolution performance and physical stability of the amorphous dispersion. Images from this analysis are provided in FIGS. 5A and B. It is seen in these images that the particles of Batches 1 and 3 do not contain any visible crystallinity, and thus corroborate the findings of PXRD and mDSC that a true single-phase amorphous dispersion was formed by TKC processing.

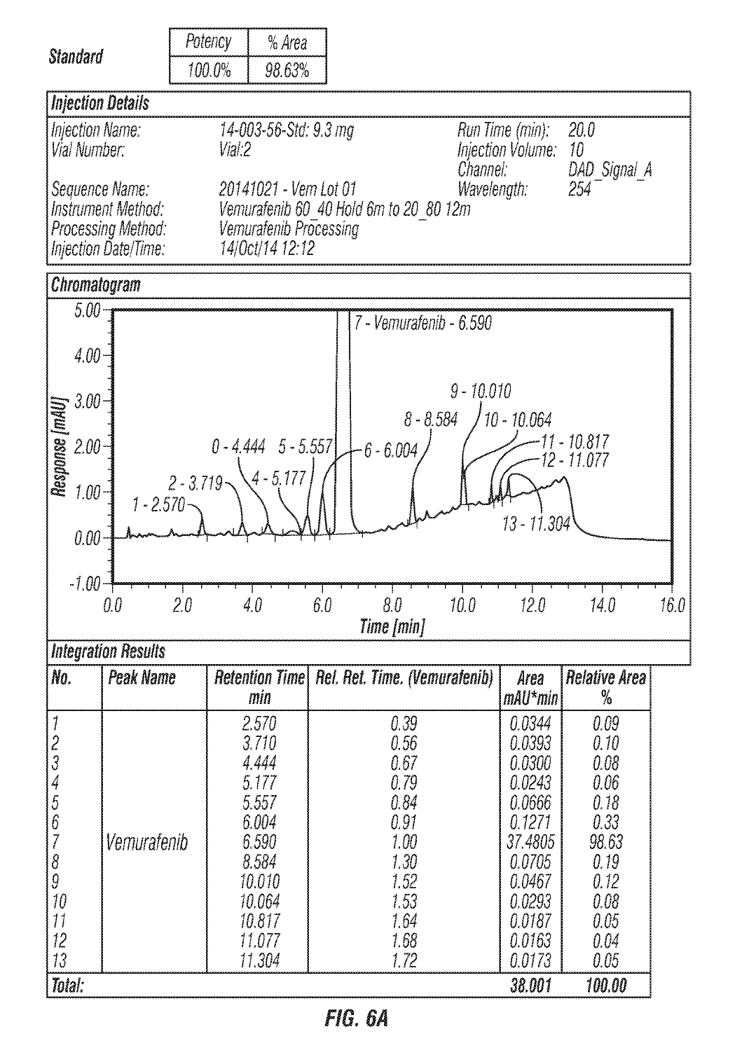

Finally, high pressure liquid chromatography analysis was performed on Batches 1 and 3 in comparison to a standard of pure vemurafenib to determine the extent of impurities formation during the TKC process. These results (FIGS. 6A and 6B) illustrate that both Batches 1 and 3 have purities of 98.6% (by relative area) which is equivalent to the pure API. Therefore, it is concluded that TKC processing generated a single-phase amorphous dispersion of vemurafenib with hypromellose acetate succinate at a 3:7 ratio with no drug degradation.

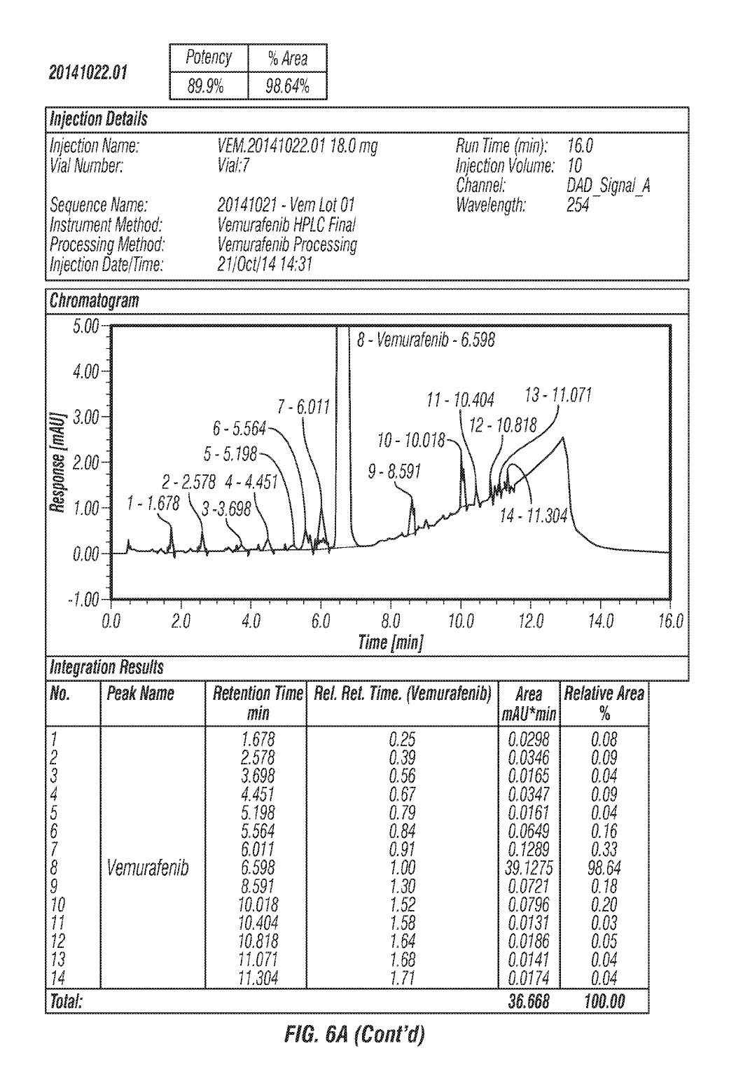

Dissolution analysis was performed on TKC Batches 1 and 3 along with MBP material to examine the release and supersaturation of each composition. USP apparatus II was utilized for dissolution testing with a fasted state pH 6.8 simulated intestinal fluid used as the dissolution media. Each sample vessel contained 500 mL of media to which amorphous intermediate containing 80 mg of vemurafenib was added. Concentration analysis was performed by high performance liquid chromatography. These results (FIG. 7) illustrate that the inclusion of SSF in the amorphous dispersion led to an improved supersaturation effect over amorphous dispersions without SSF.

### Example 2

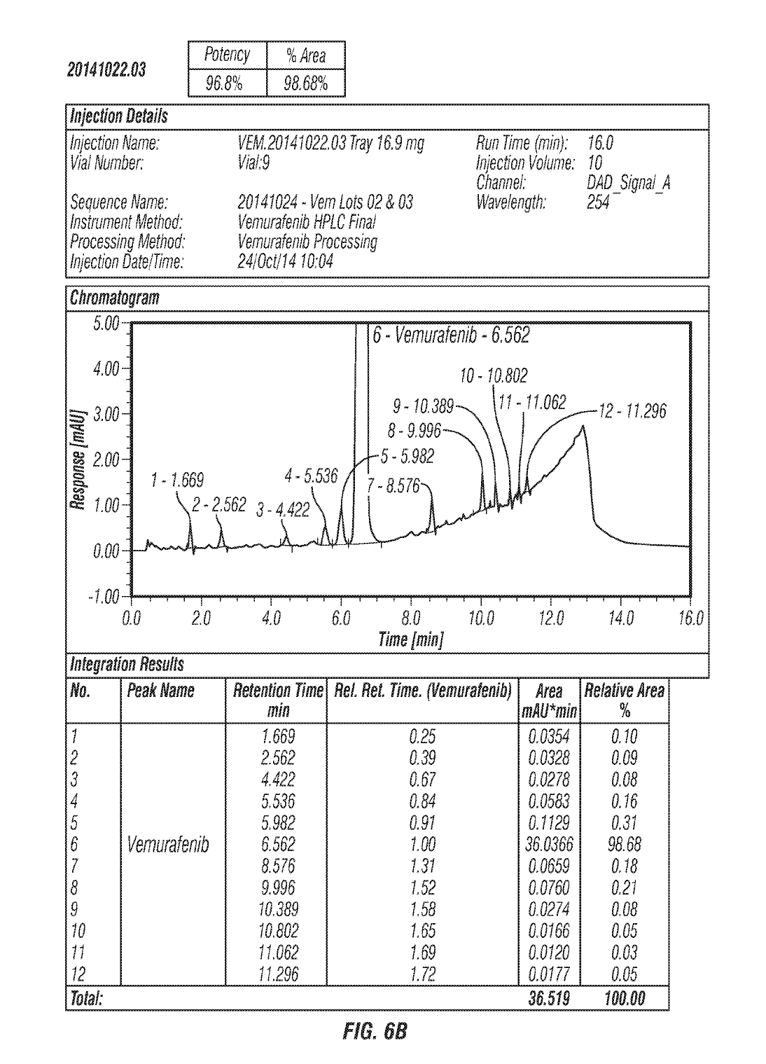

Thermokinetic compounding was performed on the compositions provided in FIG. 8. Batch VEM.20141022.04 (Batch 4) utilizes Kollidon® VA 64 (VA64) as the polymer carrier. Batch VEM.20141022.05 (Batch 5) utilizes Kollidon® VA 64 (VA64) as the polymer carrier and contains the addition of 5% dioctyl docusate sodium (DSS) as a surfactant. Batch VEM.20141022.06 (Batch 6) utilizes hydroxypropyl methylcellulose (HPMC) as the polymer carrier. Batch VEM.20141022.07 (Batch 7) utilizes hydroxypropyl methylcellulose (HPMC) as the polymer carrier and contains the addition of 5% dioctyl docusate sodium (DSS) as a surfactant.

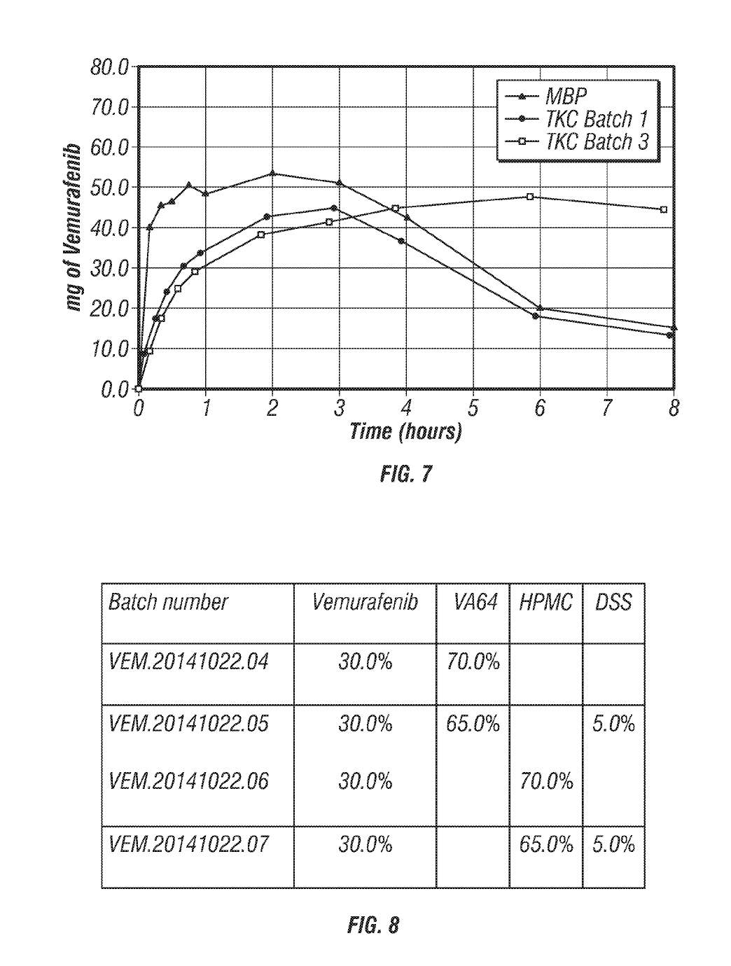

The processing parameters and temperature versus time profiles for TKC processing of Batches 4 through 7 are provided in FIG. 9. This figure signifies that the target amorphous dispersion was achieved by TKC at a peak temperature approximately 90° C. below the melting point of the API and with a time at elevated temperature of less than 10 seconds. Both the low temperature and brief processing duration are critical to producing the amorphous dispersion without degradation to the drug and/or polymer.

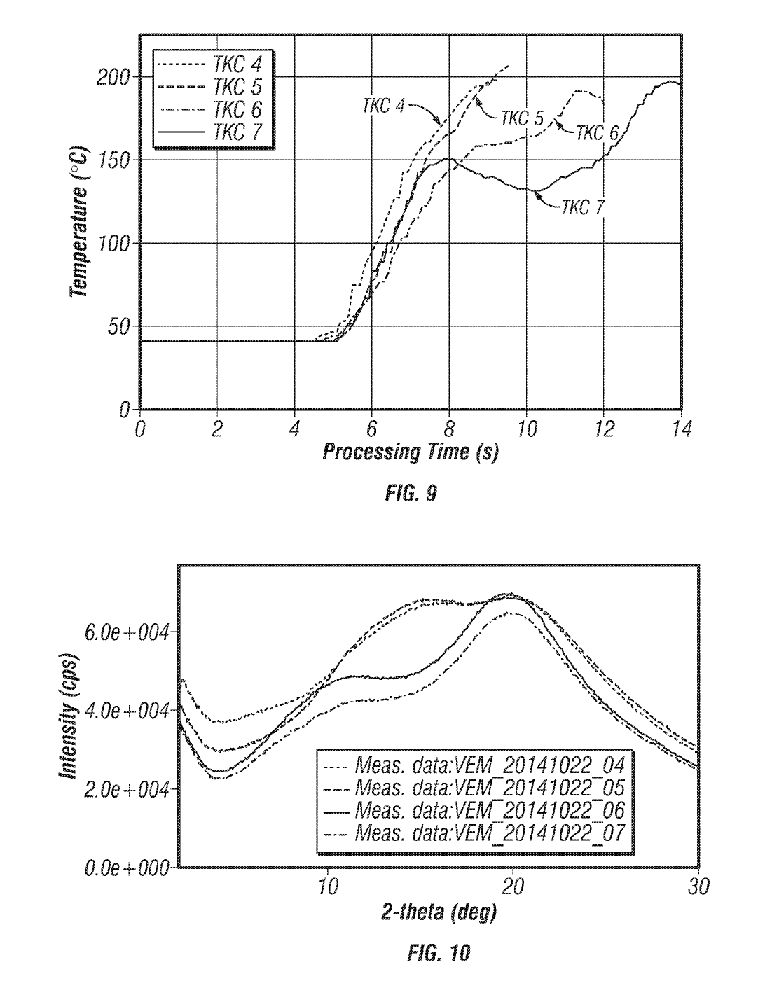

TKC Batches 4 through 7 were analyzed for crystalline content by powder x-ray diffraction (PXRD). The results of the analysis are provided in FIG. 10. These results demonstrate that Batches 4 through 7 were rendered entirely amorphous by the process (absence of drug-related crystallinity).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of making a pharmaceutical composition comprising:
(a) providing vemurafenib and one or more pharmaceutically acceptable excipients;
(b) compounding the materials of step (a) in a thermokinetic mixer for less than 300 seconds,
wherein the thermokinetic compounding of vemurafenib and the one or more pharmaceutically acceptable excipients forms a melt blended pharmaceutical composite.

2. The method of claim 1, wherein said pharmaceutical comprises one or more active pharmaceutical ingredients in addition to vemurafenib.

3. The method of claim 1, wherein the one or more pharmaceutically acceptable excipient comprises a surfactant.

4. The method of claim 1, wherein the one or more pharmaceutically acceptable excipient comprises a pharmaceutical polymer.

5. The method of claim 1, wherein the one or more pharmaceutically acceptable excipient comprises one or more surfactants and one or more polymer carriers.

6. The method of claim 1, wherein the pharmaceutically acceptable excipient comprises an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate.

7. The method of claim 4, wherein the pharmaceutical polymer comprises an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

8. The method of claim 3, wherein the surfactant comprises an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, and sorbitan laurate, and the pharmaceutical polymer comprises an agent selected from a group consisting of poly(vinylpyrrolidone), ethylacrylate-methylmethacrylate copolymer, poly(methacrylate ethylacrylate) (1:1) copolymer, hydroxypropylmethylcellulose acetate succinate, poly(butyl methacylate-co-(2-dimethylaminoethyl) methacrylate-co-methyl methacrylate) 1:2:1 and polyvinyl caprolactam-polyvinyl acetate-polyethylene glycol graft copolymer.

9. The method of claim 1, further comprising providing and compounding a MEK inhibitor with vemurafenib.

10. The method of claim 9, wherein the MEK inhibitor is GDC-0973 or cobimetinib.

11. The method of claim 1, wherein the one or more pharmaceutically acceptable excipients comprises a processing agent, such as a plasticizer.

12. The method of claim 1, wherein step (b) is performed at a maximum temperature of about 250° C., about 225° C., about 200° C., about 180° C., about 150° C. or about 150° C. to 250° C.

13. The method of claim 1, wherein the one or more pharmaceutically acceptable excipients comprises a non-ionic pharmaceutical polymer.

14. The method of claim 13, wherein the non-ionic pharmaceutical polymer is water soluble, is cellulosic, or is cellulosic and water soluble polymer.

15. The method of claim 14, wherein the non-ionic, water soluble pharmaceutical polymer is poly(vinyl acetate)-co-poly(vinylpyrrolidone) copolymer, poly(vinylpyrrolidone), hydroxypropylcellulose, poly(vinyl alcohol), hydroxypropyl methylcellulose, hydroxyethylcellulose, polyvinyl caprolactam-polyvinyl acetate-polyethylene glycol graft copolymer and sodium carboxymethyl-cellulose.

16. The method of claim 1, wherein the one or more pharmaceutically acceptable excipients comprises a cross-linked pharmaceutical polymer.

17. The method of claim 16, wherein the cross-linked pharmaceutical polymer is carbomer, crospovidone, or croscarmellose sodium.

18. The method of claim 1, wherein the one or more pharmaceutically acceptable excipients comprises a pharmaceutical polymer of high melt viscosity.

19. The method of claim 1, wherein the one or more pharmaceutically acceptable excipients comprises a thermally labile pharmaceutical polymer.

20. The method of claim 1, wherein the one or more pharmaceutically acceptable excipients comprises hypromellose acetate succinate.

21. The method of claim 1, wherein the vemurafenib to pharmaceutical polymer ratio is about 1 to 4.

22. The method of claim 1, wherein the vemurafenib to pharmaceutical polymer ratio is about 3 to 7.

23. The method of claim 1, wherein the vemurafenib to pharmaceutical polymer ratio is about 2 to 3.

24. The method of claim 1, wherein the vemurafenib to pharmaceutical polymer ratio is about 1 to 1.

25. A pharmaceutical composition comprising an amorphous dispersion of vemurafenib and one or more pharmaceutically acceptable excipients, wherein the one or more pharmaceutically acceptable excipients comprises a non-ionic pharmaceutical polymer.

26. The pharmaceutical composition of claim 25, wherein said pharmaceutical comprises one or more active pharmaceutical ingredients in addition to vemurafenib.

27. The pharmaceutical composition of claim 25, wherein the one or more pharmaceutically acceptable excipient comprises a surfactant.

28. The pharmaceutical composition of claim 25, wherein the one or more pharmaceutically acceptable excipient comprises a processing agent.

29. The pharmaceutical composition of claim 25, wherein the one or more pharmaceutically acceptable excipient comprises a plasticizer.

30. The pharmaceutical composition of claim 25, wherein the pharmaceutically acceptable excipient comprises an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate.

31. The pharmaceutical composition of claim 30, wherein the pharmaceutical polymer comprises an agent selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

32. The pharmaceutical composition of claim 25, wherein the surfactant comprises an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, and sorbitan laurate, and the pharmaceutical polymer comprises an agent selected from a group consisting of poly(vinylpyrrolidone), hydroxypropylcellulose, poly(vinyl alcohol), hydroxypropyl methylcellulose, hydroxyethylcellulose, and sodium carboxymethyl-cellulose. and polyvinyl caprolactam-polyvinyl acetate-polyethylene glycol graft copolymer.

33. The pharmaceutical composition of claim 25, wherein the pharmaceutically acceptable excipient comprises an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, sorbitan laurate, poly(vinyl acetate)-co-poly(vinylpyrrolidone) copolymer, ethylcellulose, hydroxypropylcellulose, poly(vinylpyrrolidone), poly(ethylene glycol), poly(ethylene oxide), poly(vinyl alcohol), hydroxypropyl methylcellulose, hydroxyethylcellulose, sodium carboxymethyl-cellulose, dimethylaminoethyl, and polyvinyl caprolactam-polyvinyl acetate-polyethylene glycol graft copolymer.

34. The pharmaceutical composition of claim 32, wherein the pharmaceutically acceptable excipient comprises an agent selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, sorbitan laurate, poly(vinyl acetate)-co-poly(vinylpyrrolidone) copolymer, hydroxypropylcellulose, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, and polyvinyl caprolactam-polyvinyl acetate-polyethylene glycol graft copolymer.

35. The pharmaceutical composition of claim 25, wherein said pharmaceutical composition does not contain a processing agent.

36. The pharmaceutical composition of claim 25, wherein said pharmaceutical composition does not contain a plasticizer.

37. The pharmaceutical composition of claim 25, wherein the composition is a composite and is a homogenous, heterogeneous, or heterogeneously homogenous composition.

38. The pharmaceutical composition of claim 25, wherein the non-ionic pharmaceutical polymer is a water soluble polymer.

39. The pharmaceutical composition of claim 38, wherein the non-ionic pharmaceutical water soluble polymer is selected from the group consisting of poly(vinlypyrrolidone), poly(vinyl acetate)-co-poly(vinylpyrrolidone) copolymer, hydroxypropylcellulose, poly(vinyl alcohol), hydroxypropyl methylcellulose, hydroxyethylcellulose, polyvinyl caprolactam-polyvinyl acetate-polyethylene glycol graft copolymer and sodium carboxymethyl-cellulose.

40. The pharmaceutical composition of claim 25, wherein the vemurafenib to non-ionic, water soluble pharmaceutical polymer ratio is about 1 to 4.

41. The pharmaceutical composition of claim 25, wherein the vemurafenib to non-ionic, water soluble pharmaceutical polymer ratio is about 3 to 7.

42. The pharmaceutical composition of claim 25, wherein the vemurafenib to non-ionic, water soluble pharmaceutical polymer ratio is about 2 to 3.

43. The pharmaceutical composition of claim 25, wherein the vemurafenib to non-ionic, water soluble pharmaceutical polymer ratio is about 1 to 1.

44. The pharmaceutical composition of claim 25, wherein the one or more pharmaceutically acceptable excipients comprises a pharmaceutical polymer of high melt viscosity.

45. The pharmaceutical composition of claim 25, wherein the one or more pharmaceutically acceptable excipients comprises a thermally labile pharmaceutical polymer.

46. The pharmaceutical composition of claim 25, wherein the non-ionic pharmaceutical polymer is a cellulosic polymer.

47. The pharmaceutical composition of claim 25, wherein the peak solubility of the vemurafenib in the composition is greater than 10 μg/mL in an aqueous buffer with a pH range of 4 to 8.

48. The pharmaceutical composition of claim 25, wherein peak solubility of the vemurafenib and the reference standard vemurafenib in an aqueous buffer with a pH range of 4 to 8 have a ratio of greater than 3:1.

49. The pharmaceutical composition of claim 25, wherein peak solubility of the vemurafenib and the reference standard vemurafenib in an aqueous buffer with a pH range of 4 to 8 have a ratio of greater than 10:1.

50. The pharmaceutical composition of claim 25, wherein peak solubility of the vemurafenib and the reference standard vemurafenib in an aqueous buffer with a pH range of 4 to 8 have a ratio of greater than 20:1.

51. The pharmaceutical composition of claim 25, wherein peak solubility of the vemurafenib and the reference standard vemurafenib in an aqueous buffer with a pH range of 4 to 8 have a ratio of greater than 30:1.

52. The pharmaceutical composition of claim 25, wherein in the Cₘₐₓ of the vemurafenib in the composition and Cₘₐₓ of the reference standard vemurafenib have a ratio that is greater than 6:1.

53. The pharmaceutical composition of claim 25, formulated into an oral dosage form.

54. The pharmaceutical composition of claim 53, wherein the oral dosage form is a tablet, a capsule, or a sachet.

55. The pharmaceutical composition of claim 26, wherein the second active pharmaceutical ingredient is a MEK inhibitor.

56. The pharmaceutical composition of claim 55, wherein the MEK inhibitor is cobimetinib or GDC-0973.

57. A pharmaceutical composition comprising an amorphous dispersion of vemurafenib and one or more pharmaceutically acceptable excipients, wherein the one or more pharmaceutically acceptable excipients comprises cross-linked pharmaceutical polymer.

58. The pharmaceutical composition of claim 57, wherein the cross-linked pharmaceutical polymer is carbomer, crospovidone, polycarbophil, or croscarmellose sodium.

59. A pharmaceutical composition produced by a process comprising the steps of:
(a) providing vemurafenib and one or more pharmaceutically acceptable excipients;
(b) compounding the materials of step (a) in a thermokinetic mixer for less than 300 seconds and at less than about 250° C.,
wherein the thermokinetic compounding of vemurafenib and the one or more pharmaceutically acceptable excipients forms a melt blended pharmaceutical composition.

60. The pharmaceutical composition of claim 59, wherein said one or more pharmaceutically acceptable excipients includes a non-ionic pharmaceutical polymer, a water soluble pharmaceutical polymer, cellulosic pharmaceutical polymer, a non-ionic, water soluble pharmaceutical polymer, a non-ionic, cellulosic pharmaceutical polymer, a water soluble, cellulosic pharmaceutical polymer, a thermally labile pharmaceutical polymer, a high melt viscosity pharmaceutical polymer, and/or a cross-linked pharmaceutical polymer.

61. The pharmaceutical composition of claim 60, wherein the non-ionic, water soluble pharmaceutical polymer is poly(vinlypyrrolidone), poly(vinyl acetate)-co-poly(vinylpyrrolidone) copolymer, hydroxypropylcellulose, poly(vinyl alcohol), hydroxypropyl methylcellulose, hydroxyethylcellulose, polyvinyl caprolactam-polyvinyl acetate-polyethylene glycol graft copolymer, and sodium carboxymethyl-cellulose.

62. The pharmaceutical composition of claim 61, wherein said pharmaceutical composition comprises a processing agent, such as a plasticizer.

63. The pharmaceutical formulation of claim 62, wherein said pharmaceutical composition further comprises one or more active pharmaceutical ingredients other than vemurafenib, such as a MEK inhibitor.

64. The pharmaceutical formulation of claim 62, wherein said pharmaceutical composition is combined with a co-processed with second active pharmaceutical ingredient other than vemurafenib, such as a MEK inhibitor, in a final dosage form.

65. The pharmaceutical formulation of claim 62, wherein said pharmaceutical composition is admixed with second active pharmaceutical ingredient other than vemurafenib, such as a MEK inhibitor, in a final dosage form.

66. A method of treating cancer comprising administering to a patient in need of such treatment a therapeutically effective amount of the pharmaceutical composition in claim 25.

67. The method of claim 66, where in the cancer is a solid tumor, colorectal cancer or skin cancer.

68. The method of claim 67, where in the cancer is melanoma.

69. The method of claim 68, where in the cancer is metastatic melanoma.

70. The method of claim 69, wherein the patient is BRAF V600 mutation-positive.

71. A method of treating cancer comprising administering to a patient in need of such treatment a therapeutically effective amount of the pharmaceutical formulation in claim 63.

72. The method of claim 71, where in the cancer is a solid tumor, colorectal cancer or skin cancer.

73. The method of claim 72, where in the cancer is melanoma.

74. The method of claim 73, where in the cancer is metastatic melanoma.

75. The method of claim 74, wherein the metastatic melanoma is BRAF V600 mutation-positive
